(19) United States
(12) Patent Application Publication
(10) Pub. No.: US 2023/0130034 A1
(43) Pub. Date: Apr. 27, 2023

(54) SYSTEM AND METHOD FOR PROBABILITY-BASED DETERMINATION OF STRATIGRAPHIC ANOMALIES IN A SUBSURFACE

(71) Applicant: CGG SERVICES SAS, Massy Cedex (FR)

(72) Inventors: Joseph EMMINGS, Willoughby-on-the-wolds (GB); Wenjun LEE, Llandudno (GB); Sean HUMPHREYS, Llandudno (GB); Graeme PETRIE, Old Colwyn (GB); Natalia WASIELKA, Llandudno (GB)

(21) Appl. No.: 17/894,519

(22) Filed: Aug. 24, 2022

Related U.S. Application Data
(60) Provisional application No. 63/255,667, filed on Oct. 14, 2021.

(51) Int. Cl. G01N 33/24; G01N 23/223; E21B 49/02

(52) U.S. Cl. CPC G01N 33/24; E21B 49/02; G01N 23/223; E21B 2200/20; G01N 2223/076; G01N 2223/507; G01N 2223/616

(57) ABSTRACT

A method for determining a stratigraphic anomaly in a subsurface of the earth includes receiving raw data x, assigning the rock samples to corresponding stratigraphic units of the subsurface, transforming the raw data x to a centred log-ratios clr(x) dataset, calculating p-values with a pairwise sum rank test between populations of the centred log-ratios clr(x) dataset, selecting a set of fingerprint elements from the elements of the rock samples, converting raw concentrations corresponding to the set of fingerprint elements to isometric log-ratios ilr data, determining a number of ilr sub-populations within each stratigraphic unit, applying mixture discriminant analysis to the isometric log-ratios ilr data, using the ilr sub-populations to calculate posterior probabilities of the rock samples, and identifying the stratigraphic anomaly based on the posterior probabilities.

### BACKGROUND

### Technical Field

Embodiments of the subject matter disclosed herein generally relate to methods and systems for determining stratigraphic anomalies in the subsurface and, more particularly, to mechanisms and techniques for statistical methods for determining sand injectites, local reworking, well caving, or mixing of cuttings in the subsurface.

### Discussion of the Background

Recognition of sand injectites in the subsurface is especially important. Sand injection is created through post-depositional remobilization of fluidized sands injected into overlying and neighbouring stratigraphic units, thereby forming ‘injectites’ surrounded by in-situ sedimentary rock. FIG. 1 schematically illustrates a sand injectite complex 100 extending under the Earth’s surface 102 (or ocean bottom). The sand injectite complex has a primary depositional body or parent unit 110, intrusive bodies 112, that may include sills, and dikes 114, which may extend to the earth’s surface or ocean bottom 102. The sand injectite complex may interfere/intersect with wells 120 that are being drilled for reaching an oil and gas reservoir, a sedimentary aquifer, or reservoirs for the storage of carbon dioxide 122. One or more components of the sand injectite complex may also interact with the reservoir 122. Note that the injectite complex extends over plural stratigraphic units A to E (i.e., where A to E are layers of material having common properties, each having been deposited at the same time).

A growing body of research shows sand injection is a complex phenomenon that spans a range of millimeter to decameter scales (e.g., [1]). As a result, injectites are not necessarily easy to differentiate from surrounding in-situ sedimentary rocks. Approaches to this challenge include: enhanced modern-day seismic imaging [2], field-based outcrop studies [3], and wider subsurface studies, all of which have helped to better image and identify sand injection. Injectites often have high interest in oil and gas exploration due to their high porosity and permeability [4]. As a result of these features, they are often described as “super producers.” Contrary, as the ‘injectites’ penetrate multiple overlying and neighbouring stratigraphic units (see FIG. 1), they can also act as ‘thief zones’ and breach sealing units, compromising the reservoir. Therefore, the presence and extent of injectites is directly important to a variety of industrial applications dependent on reservoirs in the subsurface, including hydrocarbon development, carbon capture and storage, geothermal energy in sedimentary basins and gas storage.

Thus, there is a need for a method for correctly identifying the presence and location of injectites in a given subsurface.

### SUMMARY OF THE INVENTION

According to an embodiment, there is a method for determining a stratigraphic anomaly in a subsurface of the earth. The method includes receiving raw data x corresponding to a geochemical elemental analysis of rock samples, wherein the raw data x is associated with elemental concentrations of elements of the rock samples, assigning the rock samples to corresponding stratigraphic units of the subsurface based on a priori data, transforming the raw data x to a centred log-ratios clr(x) dataset, calculating p-values with a pairwise sum rank test between populations of the centred log-ratios clr(x) dataset, corresponding to a given well complex, selecting a set of fingerprint elements from the elements of the rock samples, based on the p-values having a value above a given limit, converting raw concentrations corresponding to the set of fingerprint elements to isometric log-ratios ilr data, determining a number of ilr sub-populations within each stratigraphic unit, from the isometric log-ratios ilr data, using a Bayesian information criteria, applying mixture discriminant analysis to the isometric log-ratios ilr data, using the ilr sub-populations, to calculate posterior probabilities of the rock samples, and identifying the stratigraphic anomaly based on the posterior probabilities.

According to another embodiment, there is a computing device for determining a stratigraphic anomaly in a subsurface of the earth. The computing device includes an input/output interface configured to receive raw data x of geochemical elemental analysis of rock samples, wherein the raw data x is associated with elemental concentrations of elements of the rock samples, and a processor connected to the input/output interface. The processor is configured to assign the rock samples to corresponding stratigraphic units based on a priori data, transform the raw data x to a centred log-ratios clr(x) dataset, calculate p-values with a pairwise sum rank test between populations of the centred log-ratios clr(x) dataset, corresponding to a given well complex, select a set of fingerprint elements from the elements of the rock samples, based on the p-values having a value above a given limit, convert raw concentrations corresponding to the set of fingerprint elements to isometric log-ratios ilr data, determine a number of ilr sub-populations within each stratigraphic unit, from the isometric log-ratios ilr data, using a Bayesian information criteria, apply mixture discriminant analysis to the isometric log-ratios ilr data, using the ilr sub-populations, to calculate posterior probabilities of the rock samples, and identify the stratigraphic anomaly based on the posterior probabilities.

According to yet another embodiment, there is a method for determining sandstone injectites in a subsurface of the earth, and the method includes receiving X-ray fluorescence raw data x of geochemical elemental analysis of rock samples, wherein the raw data x is associated with elemental concentrations of elements of the rock samples, calculating p-values with a Wilcoxon pairwise sum rank test between populations of the raw data x corresponding to a given well complex, selecting a set of fingerprint elements from the elements of the rock samples, based on the p-values having a value above a given limit, converting raw concentrations corresponding to the set of fingerprint elements to isometric log-ratios ilr data, applying mixture discriminant analysis to the isometric log-ratios ilr data, using ilr sub-populations, to calculate posterior probabilities of the rock samples, and identifying the sandstone injectite based on the posterior probabilities.

### BRIEF DESCRIPTION OF THE DRAWINGS

For a more complete understanding of the present invention, reference is now made to the following descriptions taken in conjunction with the accompanying drawings, in which:

FIG. 1 illustrates a subsurface and an injectite complex that extends over plural stratigraphic units of the subsurface;

FIGS. 2A to 2C illustrate various stratigraphic anomalies that are present in the subsurface;

FIG. 3 is a flow chart of a method for determining one or more stratigraphic anomalies based on statistical calculations and elemental concentrations of rock samples taken from one or more wells drilled into the subsurface;

FIGS. 4A and 4B are a fingerprinting workflow used to determine the stratigraphic anomalies;

FIGS. 5A and 5B illustrate global pairwise Wilcoxon sum rank testing between stratigraphic units A to H;

FIGS. 6A to 6D summarize the pairwise Wilcoxon sum rank testing between stratigraphic units A to H for a global well complex, local well complex, across well permutation complex, and strat-by-strat complex, respectively;

FIG. 7 illustrates the Bayesian information criterion for a series of finite Gaussian mixture models versus the number of sub-populations in stratigraphic package F;

FIG. 8 illustrates sub-populations within stratigraphic package F, determined by finite Gaussian mixture modelling, visualized as biplots between each ilr value;

FIG. 9 illustrates optimized mixture discriminant analysis for stratigraphic units A-G; the ellipses show the locations and covariances of each modelled Gaussian sub-population;

FIG. 10 illustrates example sample classification obtained by applying mixture discriminant analysis;

FIGS. 11A to 11C illustrate results filtered for ‘deep-to-shallow’ pairs; each data point corresponds to a pair of stratigraphic units, predicted-observed. High probability pairs with large vertical offsets between the sampled depth and the top of the predicted stratigraphic package corresponds to the zone of injection (injectites) while high probability pairs with low vertical offsets correspond to cuttings mixing or local reworking;

FIGS. 12A to 12C illustrate results filtered for ‘shallow-to-deep’ pairs; each data point corresponds to a pair of stratigraphic units, predicted-observed;

FIG. 13 illustrates results filtered for high probability ‘deep-to-shallow’ pairs on a well-by-well basis;

FIG. 14 illustrates results filtered for high probability ‘shallow-to-deep’ pairs on a well-by-well basis;

FIGS. 15A and 15B illustrate a summary of the fractions of high probability injectite parent-daughter and parent occurrences in the considered dataset;

FIG. 16 is a flow chart of another method for determining one or more stratigraphic anomalies based on statistical calculations and elemental concentrations of rock samples taken from one or more wells of the subsurface; and

FIG. 17 is a schematic diagram of a computing device configured to perform one or more of the above-discussed methods.

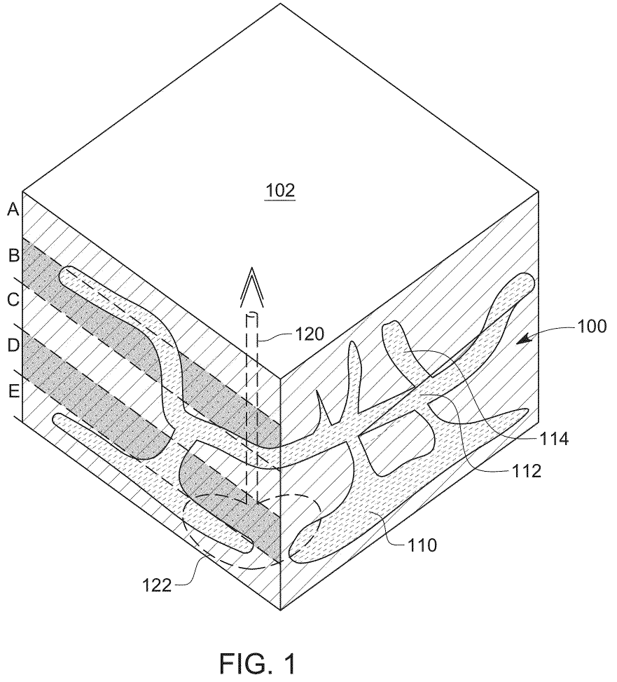

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of a land subsurface. However, the embodiments to be discussed next are not limited to a land subsurface, but may also be applied to a marine subsurface.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, there is a method that applies multivariate statistical analysis of major and trace element X-Ray fluorescence (XRF) analyses of rock cuttings or core to chemically fingerprint stratigraphic units in the subsurface of sedimentary basins. Other geochemical analysis methods, e.g., Inductively Coupled Plasma Mass Spectrometry (ICP-MS) or other spectrometric techniques may be used instead of XRF. A stratigraphic unit refers herein to a strata or layer of the subsurface that has a given set of properties (e.g., density, porosity, electrical conductance, impedance, etc.) and the subsurface is made of plural stratigraphic units.

In one application, at least one of four use-cases are envisaged to be determined/calculated by this method:
(1) detection of downward cross-contamination of shallow cuttings into deeper parts of the well, for example, due to caving of the wall of the well, which is relevant to any drilling activity within sedimentary basins or other contexts (such as mining) where layering of rock compositions is expected;
(2) detection of discrepancies between stratigraphic interpretations derived from geophysical data versus the ground-truth sample data;
(3) detection of local depositional reworking and gradational contacts between different stratigraphic units; and
(4) detection of the presence and distribution of sandstone injectites in the subsurface.

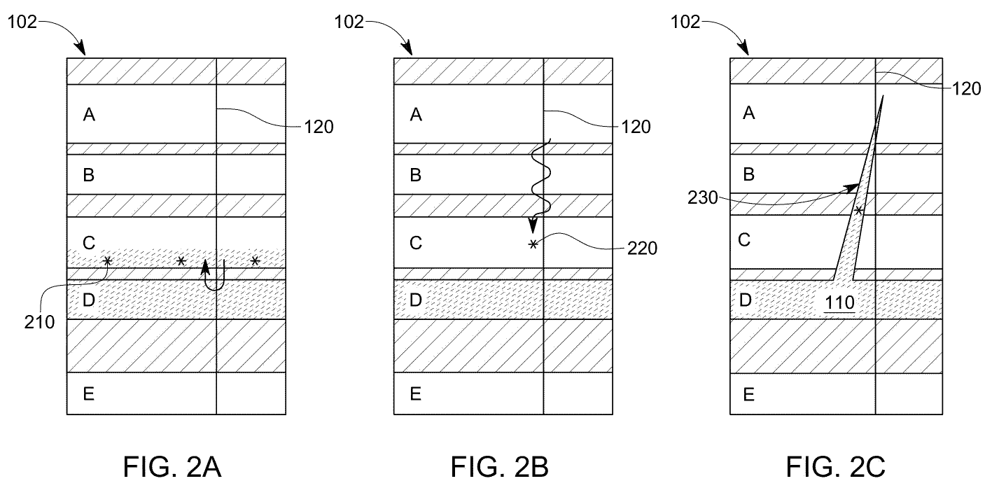

Cases (2)-(4) are particularly relevant to the hydrocarbon, geothermal and carbon capture, utilization and storage (CCUS) industries, whereas case (1) is applicable to any drilling activity through rocks. In all these applications, the proposed method quantifies - through geochemical analysis and multivariate statistics - the probability of a particular sample belonging to a different stratigraphic unit in the subsurface. For example, FIG. 2A illustrates the case when local reworking (i.e., reworking and incorporation of sand by erosion of the top of unit D during deposition of unit C) or cutting mixing during drilling is present, i.e., a sample 210 that belongs to unit D is found in unit C, or the other way around. FIG. 2B illustrates the case when caving or cross contamination is present, i.e., a sample 220 belonging to unit B is found in a different unit C, as either the walls of the well 120 have collapsed or the sample was taken during the drilling phase from unit B to unit C. FIG. 2C illustrates a third case in which the injectite 230 (i.e., the daughter) of the parent formation 110 present in unit D has been extended into unit A, due to seismicity or other geological events. For this case, the injectite 230 might cross the well 120. Several probability values are generated for each sample 210, 220 or 230, corresponding to the likelihood that the sample is more closely related (chemically) to a different stratigraphic package/unit A to D then the unit in which the sample is currently present.

Note that FIGS. 2A to 2C schematically illustrate different scenarios which may explain why the geochemical composition of a sample does not match the a priori stratigraphic framework (stratigraphic units A to E - here representing sand packages). As FIG. 2A shows local reworking or mixing of cuttings during drilling, it can explain meter-scale conflation of stratigraphic packages C and D. FIG. 2B, which shows caving or other cross-contamination (including post-drilling), may explain decameter-scale downward (i.e., shallow-to-deep) migration of rock from stratigraphic unit A into the depth of stratigraphic unit C. FIG. 2C, corresponding to sand injection, may explain decameter to 100 s-m scale upward (i.e., deep-to-shallow) migration of rock from stratigraphic unit D into C, B and A.

Case (4) is realized because the proposed fingerprinting method ranks sandstone samples in terms of their probability of being a ‘daughter’ injectite derived from a deeper sandstone unit. Inference of injection and the vertical stratigraphic reach of injection represents an important improvement that is independent of (and not constrained by) geophysical approaches. For example, the proposed method is not limited to the resolution of geophysical data (e.g., seismic data) and is able to be deployed en-masse using drill cuttings. Thus, this approach can be applied to legacy cuttings or new cuttings or rock core, and can be used to supplement existing methods, to de-risk the spatial and stratigraphic nature of injection.

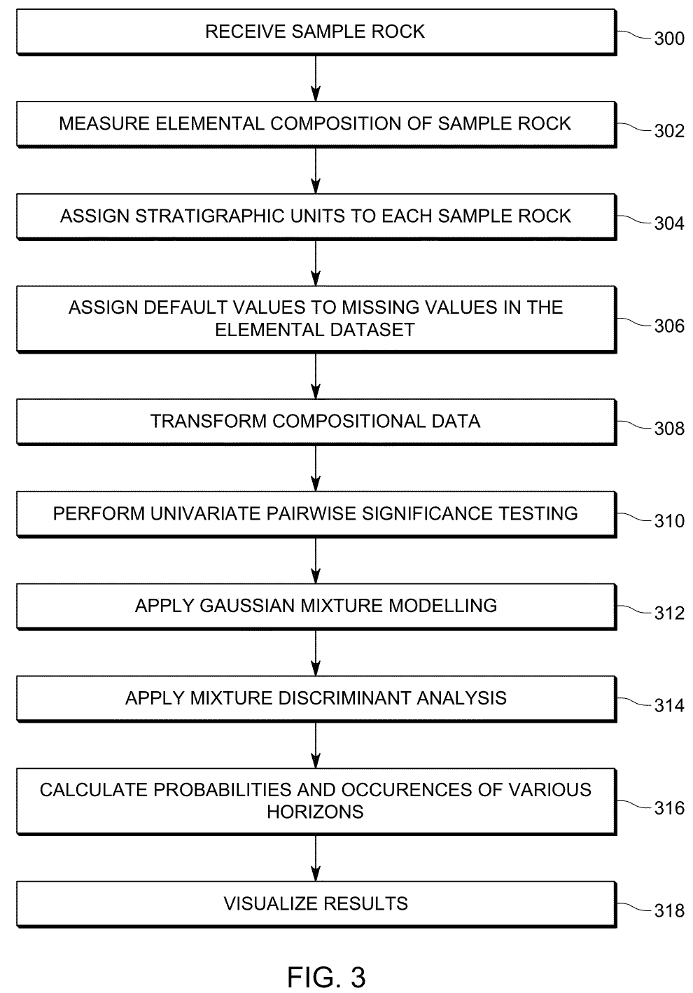

According to an embodiment illustrated in FIG. 3, a method for determining the nature and location of a stratigraphic anomaly into a subsurface starts with a step 300 of obtaining a whole rock or drill cutting samples from a well that extends into the subsurface, for example, for oil and gas exploration. The samples are then taken to an analysis device, for example, an X-Ray Fluorescence (XRF) machine for determination of major and trace element concentrations. Such a machine is capable of measuring in step 302 the elemental composition of the whole rock or drill cutting samples across a series of stratigraphic units, for a single well or multiple wells in the field. In step 304, assignment of stratigraphic units (for example, with regard to FIGS. 2A to 2C, a stratigraphic unit may be one of the layers A to D) to each sample is performed, based on a priori geological knowledge and/or supplementary datasets. For example, a seismic survey and/or prior stratigraphic survey has taken place in a given area and thus, the stratigraphic framework of that area is known. This information is the a priori geological knowledge noted above.

In step 306, any missing values in the elemental dataset are assigned a default value ahead of the statistical analysis step. In step 308, the compositional data corresponding to the rock samples is transformed (as discussed later) into one or more domains, ahead of the statistical analysis step. In step 310, univariate pairwise significance testing is performed to assist the selection of the significant fingerprint elements for each stratigraphic unit. In step 312, finite Gaussian mixture modelling is applied to the transformed compositional data, based on the fingerprint elements, to detect a number of sub-populations (clusters) within each stratigraphic unit. In step 314, a mixture discriminant analysis (MDA) is performed to determine posterior probabilities that each sample belongs to a stratigraphic unit that is not the same as the a priori assumption. Finally, in step 316, an assessment of the probabilities and occurrences of down-hole contamination (caving), local depositional reworking, cuttings mixing and post-depositional sand injection (sampling of injectites) is calculated. Based on these assessments, the method vizualizes in step 318 the results in various ways, as discussed later.

The method discussed may be applied to the following situations. Drill cuttings may become mixed within the well drilling mud over small intervals (meters to decameters) during drilling. Thus, samples with a high probability of belonging to a different stratigraphic unit within small vertical distances may represent an artefact of drilling - i.e., the mixing of cuttings across different stratigraphic units, as schematically illustrated in FIG. 2A. Alternatively, small vertical distances between the sample and the linked sandstone unit may represent true local reworking during deposition, or small discrepancies between the predicted and observed stratigraphy, as illustrated in FIG. 2B. Constraining the local reworking is desired because it (1) helps to understand the depositional processes in the sedimentary system, as an important control on reservoir quality, and (2) helps to identify incorrectly assigned stratigraphic units based on the post-drill well log interpretations.

In contrast to mixing of cuttings during drilling or reworking during deposition, which are manifested on meter scales, sand injection is often developed over decameter to 100 s m vertical distances between the sample and the linked parent sandstone unit, as illustrated in FIG. 2C. Quantification of the probability of injection and the stratigraphic reach of injection, using the proposed geochemical method, represents an important improvement that is independent of (and not constrained by) geophysical approaches. Inference of the parent sandstone unit 110 that is connected to the sampled daughter 230, and by extension estimation of minimum vertical length of injection, is desired because it (1) improves understanding of the reservoir connectivity between different sandstone units within a single well or range of wells, and (2) provides information on the number of sealing units which have been breached by a reservoir (injected) sand.

The proposed method is advantageous because it is not limited to the resolution of geophysical data (typically only capable of resolving sandstone units and injectites at decameter to 100 s meter scales), and it is able to be deployed en-masse to drill cuttings. This proposed method de-risks the spatial and stratigraphic nature of injection at an order of magnitude greater resolution than existing geophysical methods. The proposed method is also advantageous because it is robust to any alterations of the rock composition caused by sand injection itself (e.g., hydrodynamic sorting during injection). This is because this approach models relative proportions of key fingerprint elements (robust to closed data effects) and is based on natural sub-populations within each stratigraphic unit. Importantly, stratigraphic sub-populations represent the natural variability of rock compositions due to depositional hydrodynamic sorting within each stratigraphic unit. By extension, the proposed method is automatically adjusted for any hydrodynamic sorting effects caused by injection. The stratigraphic resolution of this method is limited only to size of the rock sample or the zone of mixing of cuttings within the well. In terms of injection, the proposed method can be applied to legacy or new rock core or cuttings and is relevant to drilling activities concerned with the investigation of siliciclastic reservoir quality and volume. More broadly, the assessment of cross-contamination is relevant to any drilling activity through compositionally distinct layers of rock (sedimentary, metamorphic or igneous).

Some of the steps noted above are now discussed in more detail. For example, the step 302 of measuring the elemental composition of the sample rocks may use X-Ray Fluorescence (XRF). XRF is an analytical technique used for quantifying elemental compositions of a material. When exposed to X-ray radiation, each element in the sample emits a set of characteristic secondary X-rays. As a result, XRF spectroscopy is routinely used to quantify the elemental (e.g., Na, Mg, Al, Si, etc.) abundances in each sample. By extension, by comparing the elemental composition between samples, it is possible to assign fingerprint compositions for each stratigraphic unit. In this regard, note that most sample rocks are made of a unique combination of elements, and thus, their XRF signature is unique. Compositional differences are related to lithology, depositional processes, diagenesis, and in some cases, sand injection. The method can be applied to whole rock core samples or sieved and picked sandstone fractions derived from drill cutting samples. Sieving and picking is performed to magnify the sensitivity to sand provenance (i.e., the stratigraphic fingerprint) and minimize effects related to diagenesis and/or biogenic components. The methodology has been developed using a benchtop energy dispersive XRF system capable of detecting trace elements, but this approach can be applied to major and trace element datasets acquired using different equipment (for example, inductively coupled plasma mass spectrometry, ICP-MS, etc.).

Step 304 provides a prior assignment of a stratigraphic framework. Each sample is assigned to a lithostratigraphic unit (A, B, C, etc., as shown in FIGS. 2A to 2C), commonly at the level of Formation or Member according to [5]. This information may be derived from the interpretation of geophysical data (picking the ‘tops’ of each stratigraphic unit), well logs and/or directly via biostratigraphic or chemostratigraphic analysis of the same rock samples.

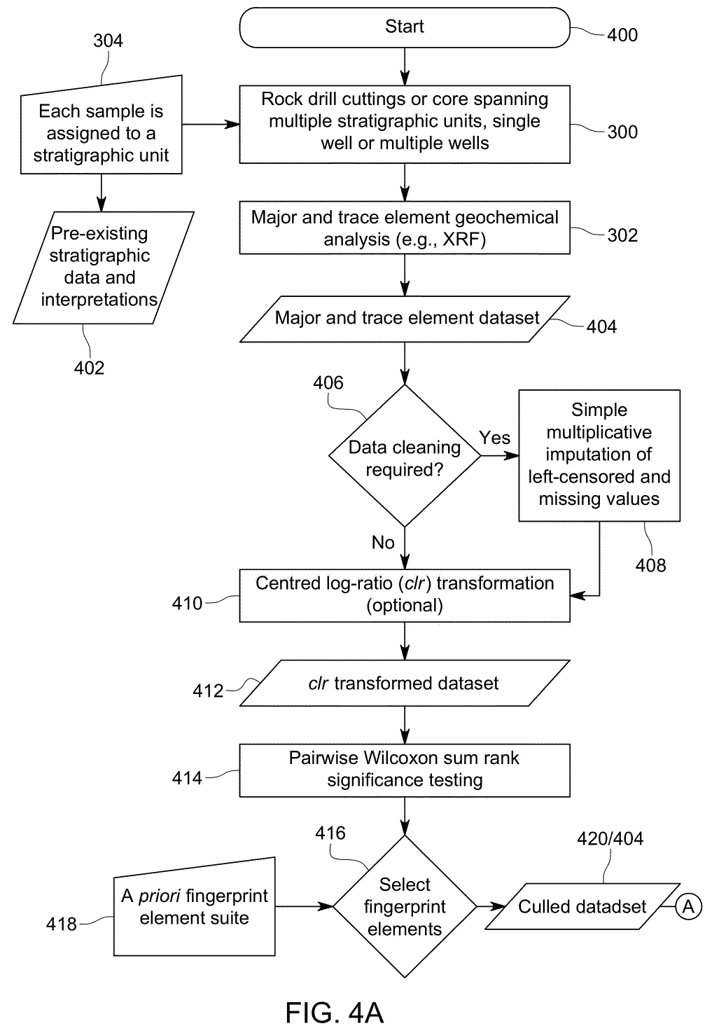

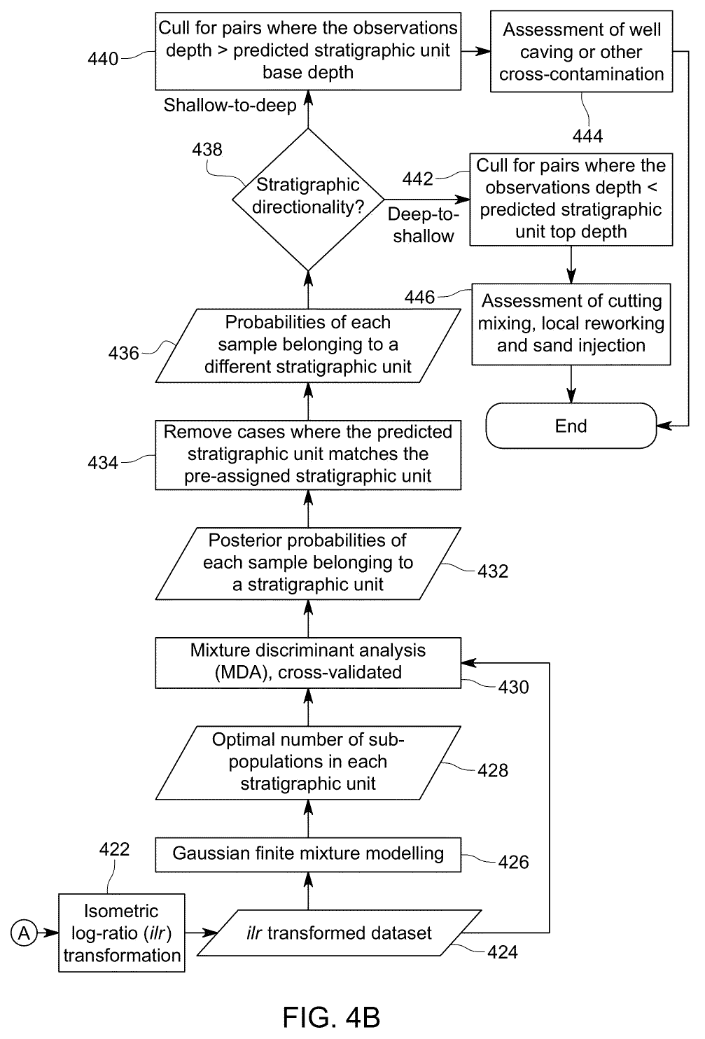

Steps 306 to 314 of the method illustrated in FIG. 3 may be considered to constitute the geostatistical workflow, which is responsible for the sand injectite identification and/or characterization. Thus, these steps are now discussed in more detail. These steps may be expressed in more detail according to the flow chart illustrated in FIGS. 4A and 4B. According to this flow chart, the method starts in step 400 where the system performing the analysis is initiated and pre-existing data 402 (e.g., stratigraphic data and interpretations) is loaded into the system. In step 300 the rock drill cuttings or core or other samples are received and analyzed in step 302, as previously discussed. While step 302 refers to XRF analysis, other elemental analysis methods may be used. In step 304, each sample is assigned to a stratigraphic unit as discussed above. As a result of step 302, major and trace element datasets 404 (or raw data x being indicative of the elemental concentrations) are determined. In step 406 it is determined whether data cleaning is necessary. This happens when values are missing or are below a detection limit for the elements of the sample rocks. If the result of this determination is yes, the method advances to step 408, for replacing left-censored elemental data (missing concentrations, or concentrations below the detection limit) using, for example, a simple replacement (e.g., 65% of the limit of detection) or non-parametric multiplicative imputation (this step corresponds to step 306 in FIG. 3). In one application, this step may use a simple multiplicative replacement of left-censored data and imputation of zero values.

Zero concentration values are not permissible due to the log transformations implemented later in this workflow. Thus, element concentrations below the limit of detection (i.e., left-censored values) are replaced in this embodiment with 65% of the reported limit of detection of the XRF analysis followed by re-closure of the dataset, using a simple multiplicative replacement method (see, for example, [6]). Other values may be used instead of the 65%. According to this approach, the transformed element concentration rⱼ for the part (element) j for a given sample is given by:

$${r}_{j} = \left\{\begin{matrix}\frac{c}{c + \left({\sum }_{k \left|{x}_{k} = 0\right)} {\hat{δ}}_{k}\right)} {\hat{δ}}_{j} , & i f {x}_{j} = 0 \\ \frac{c}{c + \left({\sum }_{k \left|{x}_{k} = 0\right)} {\hat{δ}}_{k}\right)} {x}_{j} , & i f {x}_{j} > 0\end{matrix}\right)$$

where δ is the imputed concentration for the element j, x is the initial element j concentration in each sample, k refers to other parts (e.g., elements) of the composition, c is the constant of the sum constraint (up to 10⁶ in the case of XRF data, where concentrations are in parts per million), and δ is the imputed concentration for element j. Thus, in this step it is possible to assign 65% of the reported limit of detection for element j if there is a missing value for element j.

Alternatively, to better preserve ratios, the replaced left-censored values (if present) are excluded from the re-closure operation (see, also [6]), i.e.,

$${r}_{j} = \left\{\begin{matrix}\text{} \text{} {δ}_{j} , & i f {x}_{j} = 0 \\ \left(1 - \frac{{\sum }_{k \left|{x}_{k} = 0\right)} {δ}_{k}}{c}\right) {x}_{j} , & i f {x}_{j} > 0\end{matrix}\right)$$

Finally, missing values (if present) are imputed using the geometric mean of the element concentrations in the dataset, followed by an optional re-closure (see [6]), i.e.,

$${r}_{j} = \left\{\begin{matrix}{m}_{j} , & i f {x}_{j} i s m i s s i n g \\ \frac{\left(c - {\sum }_{k \left|{x}_{k}\right)} {m}_{k}\right)}{{\sum }_{k \left|{x}_{k}\right)} {m}_{k}} {x}_{j} , & i f {x}_{j} i s n o n m i s s i n g\end{matrix}\right)$$

$$w h e r e {m}_{j} = {\left({\prod }_{i = 1}^{n} {x}_{i j}\right)}^{1 / n}$$

and where, m is the geometric mean of j element across the sample suite and n is the number of samples in the dataset.

Next, the process moves to step 410 for transforming raw element concentrations (from dataset 404) into centred log-ratios (clr), which results in a clr transformed dataset 412. The XRF compositional data 404 is closed by a sum constraint of 10⁶ ppm or 100%. A centred log-ratio (clr) transformation is applied to the dataset to extract the relative relationships between elements (e.g., fingerprinting) and to avoid artificial closed data effects related to changes in the bulk mineralogy. More specifically, the clr transformation is given by:

$$c l r \left(x\right) = \left[ln \left({x}_{D}\right) / g \left(x\right)\right] ,$$

$$w i t h g \left(x\right) = {\left({\prod }_{i = 1}^{D} {x}_{i}\right)}^{1 / D}$$

where x is a composition (i.e., sample) comprising D elements and g(x) is the geometric mean of the composition x across D elements.

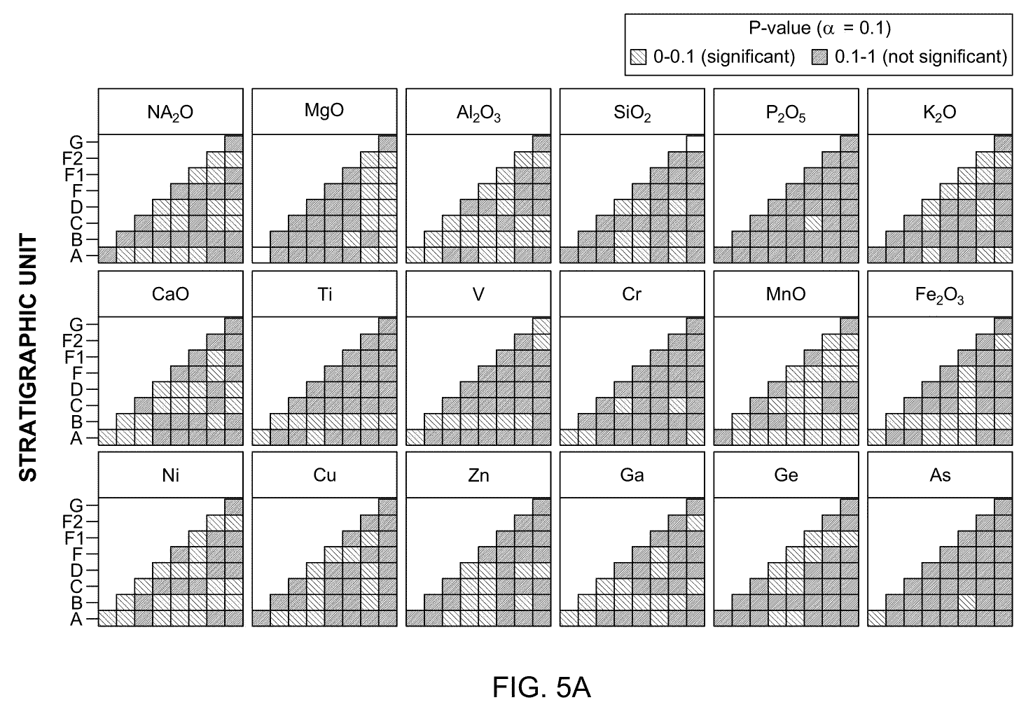

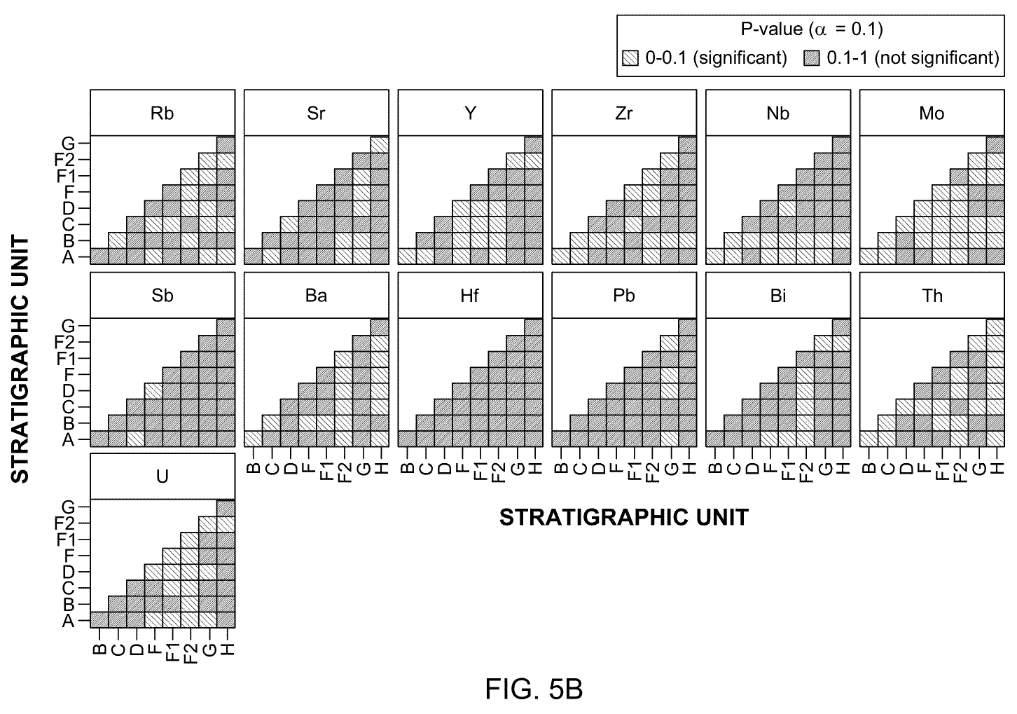

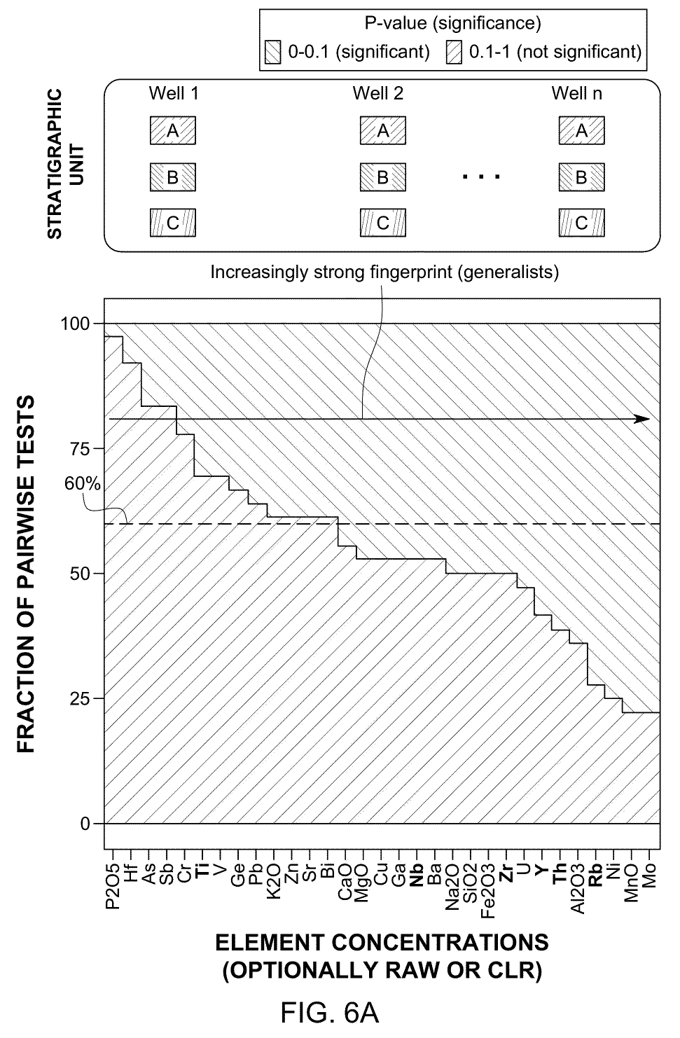

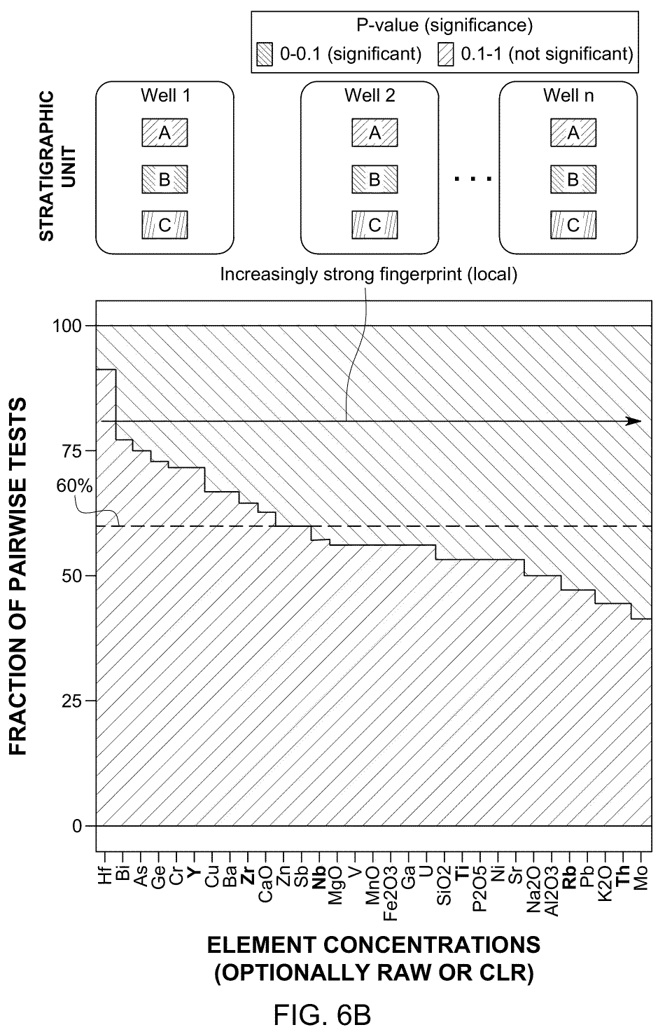

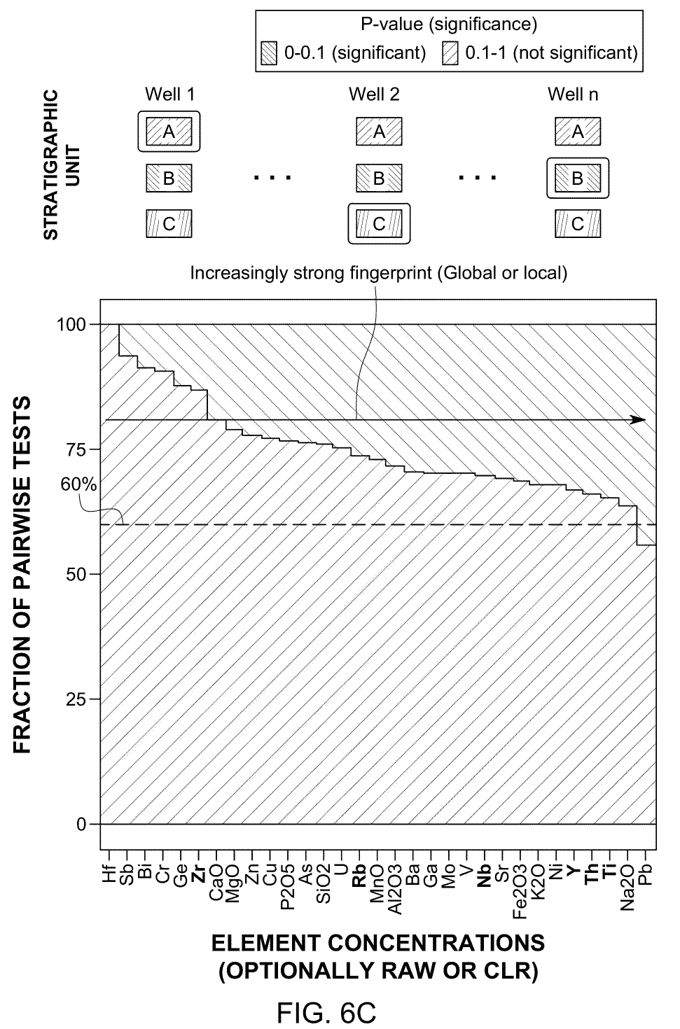

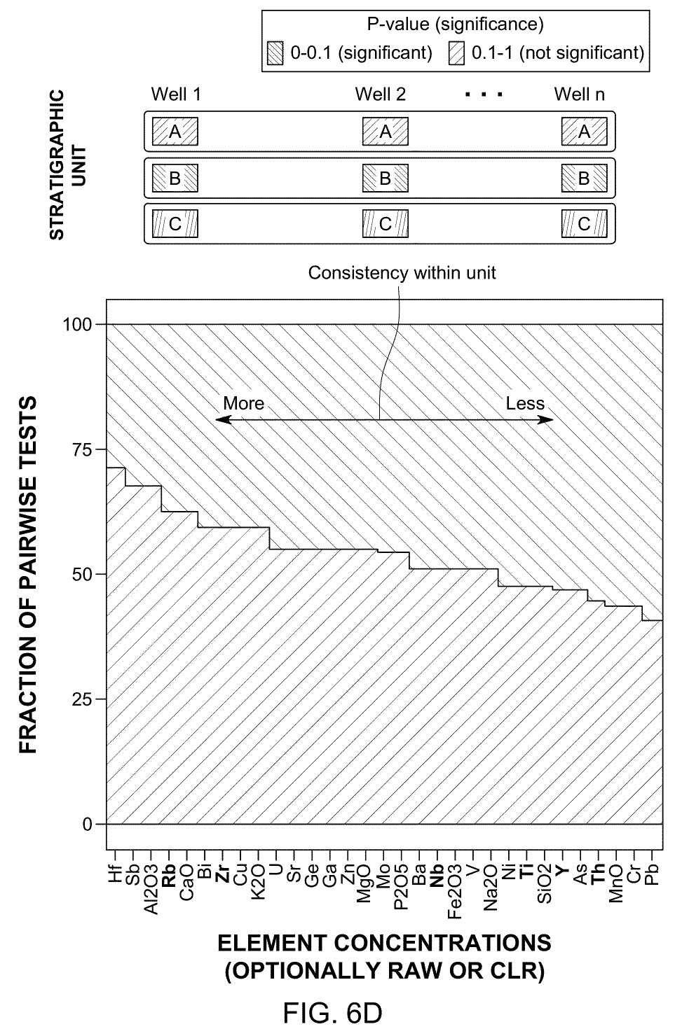

Next, in step 414, the clr-transformed values are grouped according to the a priori stratigraphic unit (definition of ‘population’), followed by application of pairwise Wilcoxon significance testing between each stratigraphic unit population, for (a) all wells in a given field (i.e., a ‘global’ workflow), (b) well-by-well (i.e., a ‘local’ workflow), and (c) within each stratigraphic unit in turn (‘strat-by-strat’). FIGS. 5A and 5B are an example of ‘global’ pairwise Wilcoxon sum rank testing results between stratigraphic packages A-H. The p-values from the Wicoxon testing (as shown in FIGS. 5A and 5B) at a given α value are calculated and aggregated in this step. These values may be presented, as illustrated in FIGS. 6A to 6D, for the global approach, local approach, across well permutation, and the strat-by-strat approach, respectively. FIGS. 6A to 6D plot the fraction of pairwise tests versus the various elements and compositions that are expected to be found in the subsurface. Note that the top of each of the FIGS. 6A to 6D schematically illustrates how the wells and/or units from various layers are considered when performing these calculations. Also note that elements at the bottom of the graphs (dark grey) represent a priori selected fingerprint elements.

In step 416, key elements for multivariate analysis (the sub-composition of elements) are selected so that corresponding p-values are less than a given limit (e.g., a value of 0.1), i.e., omitting elements which exhibit low proportions of significant global and local Wilcoxon test p-values, and also omitting the elements which are not considered to be credible fingerprints for stratigraphic units based on prior geological knowledge.

This step may be implemented as follows. The XRF analysis of rock commonly yields the concentrations 404 of about 30-50 elements, but most elements derive from multiple, potentially non-unique sources in any given depositional and diagenetic system. Thus, only a small number of elements offer a means of robust spatiotemporal fingerprinting (chemostratigraphy). The author in [7] defines a chemostratigraphic fingerprint as “the unique rock record defined by chemostratigraphic indices and recognizable through unique geochemical signature(s) which in turn helps distinction of a designated rock record from other rock records and also correlation with applicable analogues at appropriate/applicable spatiotemporal scale.” Key fingerprint elements (the sub-composition) are initially selected based on prior, geological knowledge database 418. The method of FIGS. 4A and 4B utilizes (but is not limited to) Ti, Zr, Y, Rb, Nb and Th as the elements that belong to the sub-composition, as these elements are relatively immobile in the diagenetic systems and partition preferentially into the heavy mineral (sand) fraction. The method is flexible and capable of assessing any number of elements. Different elements may be selected where this is justified based on geological understanding of the sedimentary system under investigation.

The Wilcoxon rank sum significance test, also known as the Mann-Whitney U test, is a non-parametric statistical test that is used to determine whether the difference between two populations (of elements) is significant. In this embodiment, the Wilcoxon test is applied to each element (in the dataset 412) in turn, comparing populations of elements at the stratigraphic unit and/or well-level. The test is applied to raw or clr-transformed datasets.

In this regard, suppose X and Y are two sets of clr-transformed element concentrations randomly sampled from two populations of possible elements. The inventors tested the following hypotheses:

Null Hypothesis: H₀: X = Y, there is no significant difference between population X and population Y.

Alternate Hypothesis: H₁: X ≠ Y, there is a significant difference between population X and population Y.

The test statistic U is computed as the following:

$$U = {\sum }_{i = 1}^{{n}_{x}} {\sum }_{j = 1}^{{n}_{y}} S \left({X}_{i} , {Y}_{j}\right) ,$$

$$S \left(X , Y\right) = \left\{\begin{matrix}1 , i f X > Y , \\ \frac{1}{2} , i f X = Y , \\ 0 , i f X < Y .\end{matrix}\right)$$

where nₓ and n_{y} are the number of samples in populations X and Y.

The statistic U is standardized to a standard normal distribution enabling computation of z scores and p-values. The p-values indicate the result of the hypothesis test according to a defined significance level α. In this workflow, the pairwise test is repeated for any a values of interest, including (but not limited to) a = 0.01, 0.05 and 0.1. The z score is defined as:

$$z = \frac{U - {m}_{U}}{{σ}_{U}} ,$$

where

$${m}_{U} = \frac{{n}_{x} {n}_{y}}{2} , a n d$$

$${σ}_{U} = \sqrt{\frac{{n}_{x} {n}_{y} \left({n}_{x} + {n}_{y} + 1\right)}{12}} .$$

For example:
Let the significance level be α = 0.1.
The null hypothesis H₀ is not rejected if p < 0.1, meaning there is a significant difference between population X and population Y.
The null hypothesis H₀ is rejected if p > 0.1, meaning there is no evidence of significant difference between population X and population Y.
The Wilcoxon tests were applied to assess the significance of the populations of each fingerprint element, between different stratigraphic units and/or across wells using the following approaches:
   (a) ‘global’ significance testing (FIG. 6A), where a single stratigraphic unit is tested pairwise against another stratigraphic unit across all wells in the study area/field (see FIGS. 5A and 5B). For example, X = Stratigraphic unit A in all wells, and Y = Stratigraphic unit B in all wells.
   (b) ‘local’ well-by-well significance testing (FIG. 6B), where a single stratigraphic unit is tested pairwise against another stratigraphic within the same well. For example, X = Stratigraphic unit A in Well 1, and Y = Stratigraphic unit B in Well 1.
   (c) across well permutation significance testing (FIG. 6C), where a single stratigraphic unit in a well is tested pairwise against another stratigraphic unit from another well. For example, X = Stratigraphic unit A in Well 1, and Y = Stratigraphic unit B in Well 2.
   (d) strat-by-strat significance testing, where a single stratigraphic unit in a well is tested pairwise against the same stratigraphic unit from another well. For example, X = Stratigraphic unit A in Well 1, and Y = Stratigraphic unit A in Well 2.

Given that XRF data 404 produces a wide range of geochemical elemental data (typically > 30-50 elements), utilizing all elemental data is ineffective, inefficient and subject to competing process drivers (such as depositional processes, closed and open system diagenesis, drilling contamination). The algorithm associated with this test ranks the elements in terms of ‘power’ as a unique fingerprint for each stratigraphic unit. This approach is conducted, in one application, on a (1) ‘global’ basis, where the algorithm finds good ‘generalist’ elements which fingerprint stratigraphic units at the regional scale, and (2) ‘local’ basis, where the algorithm finds the best fingerprints on a well-by-well basis, an approach that is tuned to local depositional and/or diagenetic effects. Other implementations may be used.

For each of the four approaches (a) to (d) discussed above, the total count of significant pairs (Reject Null Hypothesis) and no evidence of significant pairs (Fail to Reject Null) at a = 0.10 (for example) were tabulated by each element, as shown in FIGS. 6A to 6D. For visualization, the results are sorted monotonically in increasing order of the fraction of significant tests.

The purpose of pairwise significance testing is to detect fingerprint elements for each stratigraphic unit, i.e., elements which exhibit significant differences between populations across stratigraphic units and/or wells. Elements which exhibit a large fraction of significant results for case (a) are generalist fingerprints that effectively discriminate each stratigraphic package irrespective of spatial location (well). Elements which exhibit a large fraction of significant results for case (b) are highly variable on a global perspective, but are effective local fingerprints for stratigraphic unit on a well-by-well basis. Elements which exhibit a large fraction of significant results for case (a) tend to exhibit a large fraction of insignificant results via case (b), and vice versa, a finding that is consistent with generalist elements being less susceptible to fractionation and ponding within local depocenters. Cases (c) and (d) are auxiliary approaches designed to ensure comprehensive understanding of each element in the system.

In the proposed method of FIGS. 4A and 4B, the pairwise significance tests are used to verify the a priori element suite 418 (for example, Ti, Zr, Y, Rb, Nb and Th). A cut-off value is assigned according to the proportion of number count of significance. For example, in the proposed method, elements with more than 40% significant pairs were adopted as a cut-off value to verify the fingerprint elements, to be utilized in the subsequent multivariate analysis. Elements with a low proportion of significant results, and/or which are known to derive from multiple, non-unique sources in the depositional and/or diagenetic system are not included in the culled dataset 420, which is subsequently used in multivariate analysis.

The next step 422 converts the raw concentrations corresponding to (1) the elements from the raw dataset 404 (see FIGS. 6A to 6D), for the selected fingerprints elements from step 416, or from (2) the elements of the clr-dataset 420, into an isometric log-ratios (ilr) dataset 424. An ilr-transform is applied to the raw dataset 404 or the clr-dataset 420 to enable analysis by classical multivariate methods. The ilr transformation in step 422 maps a composition (x) to a D-1 dimensional Euclidian vector (where D is the number of elements), according to equation:

$$i l r \left(x\right) = {V}^{t} c l r \left(x\right)$$

$$w i t h V ∈ {R}^{D \times \left(D - 1\right)} ,$$

where V is a triangular Helmert matrix (as originally defined by [8]).

Like the clr transformation, the ilr transformation enables analysis of a sub-composition of interest whilst negating closed data effects. The ilr transformation enables analysis of the relative proportionality between selected fingerprint elements that is independent of the absolute abundance of the heavy mineral fraction. This is desired in this method because the process of injection induces changes in the bulk mineralogy. The result of the ilr transformation is the ilr transformed dataset 424.

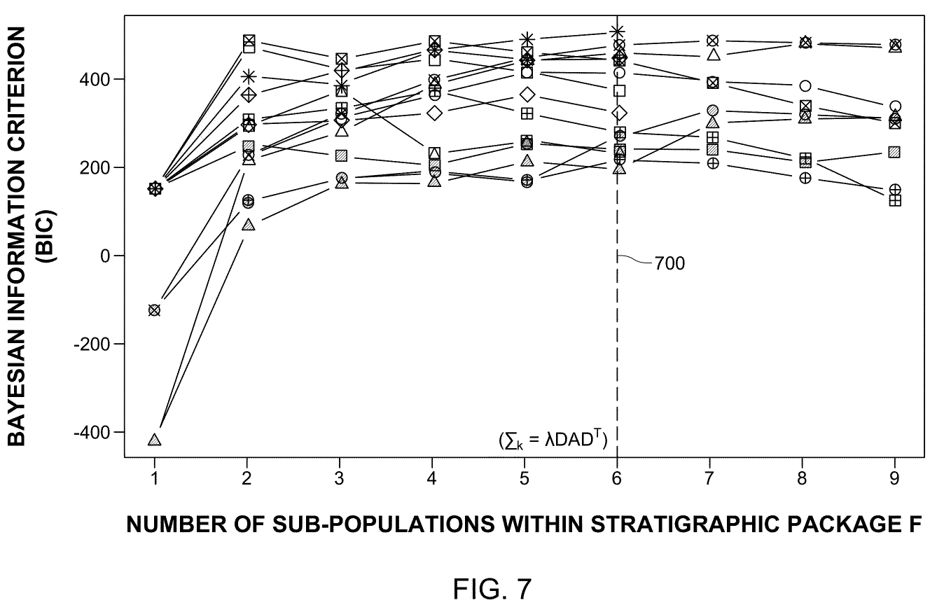

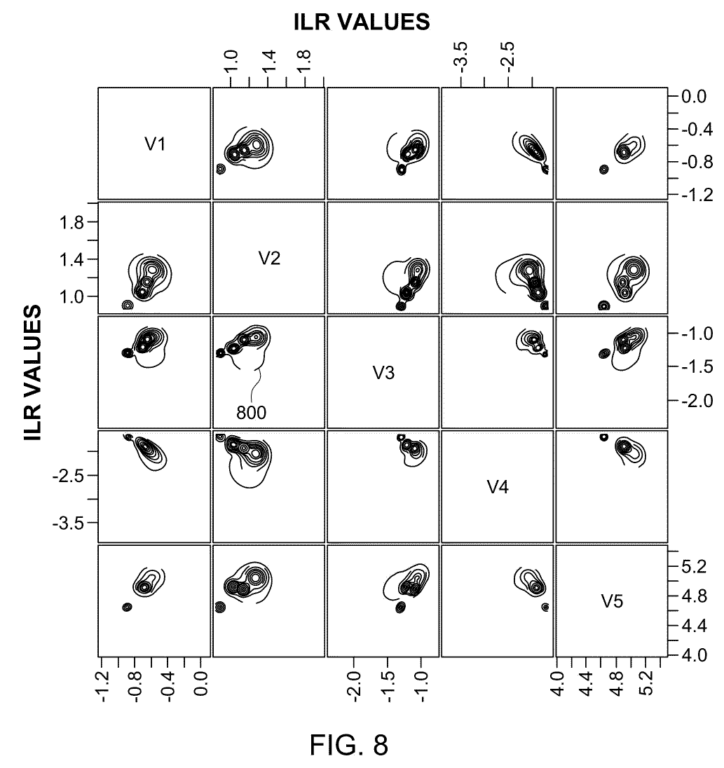

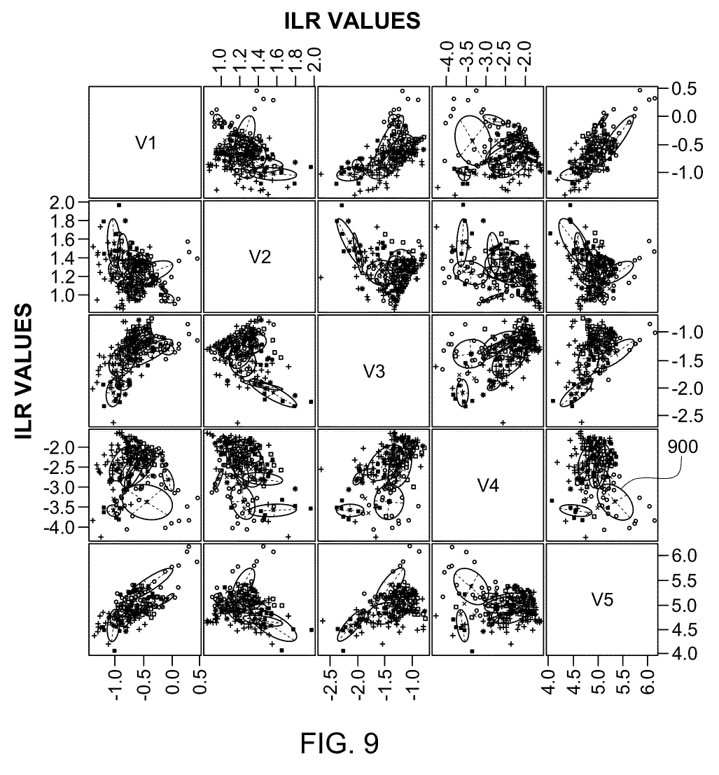

With this dataset, the optimal/best number of ilr sub-populations 428 is determined in step 426 within each stratigraphic unit. The optical number of sub-populations is determined by taking the largest Bayesian information criterion (BIC) value for a range of sub-populations and spherical and diagonal finite Gaussian mixture models, as illustrated in FIGS. 7 and 8. FIG. 7 shows the BIC for a series of finite Gaussian mixture models versus the number of sub-populations in the stratigraphic package F. An optimized solution corresponds to 6 sub-populations 700 with ellipsoidal distribution, variable volume an equal shape and orientation. Each model in FIG. 8 represents discrete permutations of the Gaussian mixture model hyperparameters. FIG. 9 represents sub-populations within the stratigraphic package F, determined by finite Gaussian mixture modelling, visualized as biplots 800 between each ilr value.

The Gaussian finite mixture modelling (see, e.g., [9] or [10]) is applied to the ilr population dataset 424 for each stratigraphic unit in turn, to determine the optimal number of sub-populations 428 present within each overall population (6 for the example shown in FIG. 8). The sub-populations are expected because some elements are not valid global fingerprints but are significant local fingerprints for stratigraphic unit. This is consistent with geological processes; spatiotemporal variation in the distribution of elements is expected, due to processes such as hydrodynamic sorting. A probability density distribution of each stratigraphic unit is approximated through a finite Gaussian mixture model.

In this regard, let

$$X = {\left\{{x}_{i} , … , {x}_{n}\right\}}_{i = 1}^{N} .$$

with xᵢ ∈ R^{D}, where N is the total number of samples from a single stratigraphic unit, D is the dimension of the elemental data after ilr transformation, and X is a collection of samples from a single stratigraphic unit. The probability density distribution f is described as the following:

$$X ∼ f \left(π , μ , Σ\right) = {\sum }_{k = 1}^{K} {π}_{k} G \left({μ}_{k} , {Σ}_{k}\right) ,$$

$${\sum }_{k = 1}^{K} {π}_{k} = 1.$$

Each of the Gaussian distributions in the mixture model is parametrized by a mean vector µₖ and covariance matrix Σₖ. The Estimation-Maximization (EM) algorithm may be used to solve for the optimal π,µ, and Σ that best represent population X.

In the Gaussian mixture modelling, there are two hyperparameters that control the fitness of the model f on X. The first parameter K represents the number of Gaussian distributions to be included in the mixture model. The second parameter Ψ is the constrained on the covariance matrix of each individual Gaussian distribution Σₖ. The Σₖ is eigen-decomposed as following:

$${Σ}_{k} = {λ}_{k} {D}_{k} {A}_{k} {D}_{k}^{T} ,$$

where λₖ is a scalar that controls the volume of the Gaussian, Aₖ is a diagonal matrix that determines the density of the Gaussian contours, and satisfies the relationship det Aₖ = 1, Dₖ is an orthogonal matrix that control the orientation of the ellipsoid (see, for example, [10]), and Ψ defines the combination of the aforementioned parameters in order to define different models for Gaussian volumes, shapes and orientations.

The best values of the hyperparameters K and Ψ that best characterize the population X are approximated via the highest scoring BIC, as illustrated in FIGS. 7 and 8. More specifically, the best values are determined based on the relationship:

$$B I {C}_{K , ψ} = 2 l o g \text{f} \left(\text{X} \left|\hat{θ} , \text{K,} ψ\right)\right) - v l o g \left(n\right)$$

where

$$\hat{θ}$$

is the estimated model parameter (comprising π, µ and Σ), n is the number of samples from X population, and v defines the number of estimated parameters in the model parameter

$$\hat{θ} .$$

The best values for K, Ψ and

$$\hat{θ}$$

define the fitting of Gaussian curves for each sub-population 800 (see FIG. 8) and are used in the subsequent mixture discriminant analysis.

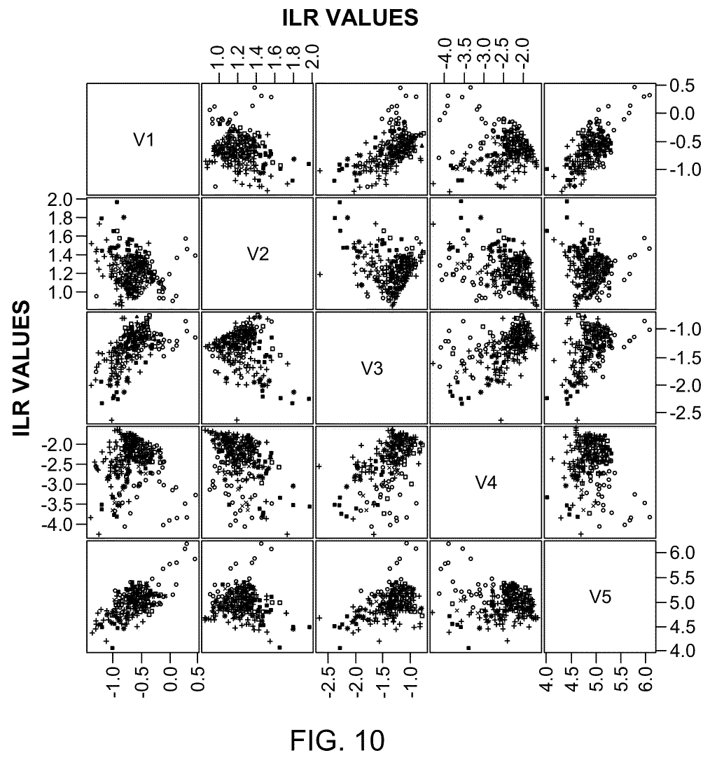

The method then advances to step 430, where a mixture discriminant analysis (MDA) is applied for the ilr-transformed dataset 424 using the optimized sub-populations 428. For this reason, the MDA box has two inputs in FIG. 4B. A posterior probability 432 of each sample belonging to a different stratigraphic unit, and the model accuracy, is determined by K-fold cross validation (CV) as illustrated in FIGS. 9 and 10. FIG. 9 illustrates the MDA for stratigraphic units A-G. The ellipses 900 show the locations and covariances of each modelled Gaussian sub-population. FIG. 10 shows example sample classification via MDA.

The MDA provides a means to quantify the probability of a given sample belonging to different classes. Thus, it provides a means to assess mixing across the observed stratigraphy, for example cuttings mixing or other cross-contamination of the samples, reworking during deposition and post-depositional injection of sand into overlying stratigraphic units.

In this regard, let

$$X = {\left\{{x}_{i} , … , {x}_{n}\right\}}_{i = 1}^{N} ,$$

with xᵢ ∈ R^{D}, where N is the total number of samples, D is the dimension of X after ilr transformation, and X is a series of elemental analyses from C number of stratigraphic units. MDA is used to build a classification model f(X) → Y ∈ {1, ..., C} and subsequently compute P(Y = c|X = xᵢ), the probability that sample xᵢ belongs to a stratigraphic unit c (see, for example, [11]). The overall model can be expressed as follows:

$${\underbrace{P \left(Y = c \left|X = {x}_{i}\right)\right)}}_{P o s t e r i o r} ∝ {\underbrace{P \left(Y = c\right)}}_{Pr i o r} {\underbrace{P \left(X = {x}_{i} \left|Y = c\right)\right) .}}_{L i k e l i h o o d}$$

$$w i t h \hat{y} = a r g m a {x}_{c ∈ \left\{1 , … C\right\}} P \left(Y = c\right) P \left(X = {x}_{i} \left|Y = c\right)\right) .$$

The equations above show that the MDA generates two components for the posterior probability, the prior probability, and the likelihood. The classification outcome ŷ is described as the stratigraphic unit c that gives highest value of the product between prior and likelihood (the posterior probability), as illustrated in FIG. 10.

For C number of stratigraphic units, this step computes the prior probability of each stratigraphic as follows:

$$P \left(Y = c\right) = \frac{{\sum }_{i = 1}^{N} 1 \left({y}_{i} = c\right)}{N} ,$$

where N is the total number of samples in the training data and the prior probability P(Y = c) is the proportion of training samples that belongs to class k in the training data.

The “likelihood” P(X = xᵢ | Y = c) can be defined as follows: given the probability density function f_{c} that represents a stratigraphic interval c, what is the likelihood of observing the sample xᵢ? In MDA, the probability density function f_{c} is represented by the Gaussian Mixture Model that was described with regard to step 426. For each stratigraphic interval, c is represented by f_{c}, which is defined as a mixture of Gaussian distributions that is parametrized by π,µ and Σ. Therefore, the likelihood of a sample xᵢ belonging to a stratigraphic interval k is computed as follows:

$$\begin{matrix}l o g P \left(X = {x}_{i} \left|Y = c\right)\right) = l o g \left({f}_{c} \left({x}_{i} \left|π , μ , Σ\right)\right)\right) \\ = l o g \left({\sum }_{g = 1}^{G} {π}_{g} \left(\frac{1}{{\sqrt{2 π}}^{D} \sqrt{\left|Σ\right|}} {e}^{{\left({x}_{i} - {μ}_{g}\right)}^{T} {Σ}^{- 1} \left({x}_{i} - {μ}_{g}\right)}\right)\right) .\end{matrix}$$

The posterior probability is proportional (see equation (12)) to the product of the prior probability and the likelihood, which means that:

Posterior Probability

$$\begin{matrix}= l o g \left(\frac{{\sum }_{i = 1}^{N} 1 \left({y}_{i} = c\right)}{N}\right) \\ + l o g \left({\sum }_{g = 1}^{G} {π}_{g} \left(\frac{1}{{\sqrt{2 π}}^{d} \sqrt{\left|Σ\right|}} e {\left({x}_{i} - {μ}_{g}\right)}^{T} {Σ}^{- 1} \left({x}_{i} - {μ}_{g}\right)\right)\right) .\end{matrix}$$

To present the posterior probability between 0 to 1, a softmax function is applied to the predicted posterior probability of all classes, where y ∈ ℝ^{C} is a vector with C dimensions, containing the posterior probabilities of class i at the i-th element of vector y. The softmax function is computed as follows:

$$N o r m a l i z e d P o s t e r i o r P r o b a b i l i t y = \frac{{e}^{{y}_{i}}}{{\sum }_{i}^{C} {e}^{{y}_{i}}} .$$

In this step, a cross-validation substep may be implemented. The purpose of cross-validation is to avoid overfitting. In this workflow, the MDA is deployed in K-fold cross-validation (CV). The dataset is split into K folds (including, but not limited to, K = 10). K-1 folds (i.e., 90% of the dataset) are used for training and the remaining fold is used for validation (10% of the dataset in this case). The folds are rotated so that each fold is used once for validation. The normalized posterior probabilities P(Y = c|X = xᵢ) that each observation in the validation fold belongs to each and any stratigraphic unit are extracted from the model via Bayes theorem. The MDA modelling is iterated (for example n = 20) to constrain the accuracy, expressed by mean and standard deviation posterior probabilities for each sample belonging to each stratigraphic unit.

In step 434, the method retains the stratigraphic pairs that define shallow samples (daughters 230) belonging to the underlying stratigraphic units (parents 110) “deep-to-shallow,” or retain the stratigraphic pairs that define deep samples belonging to overlying stratigraphic units “shallow-to-deep.” In other words, this step removes cases where the MDA-predicted stratigraphic unit matches the stratigraphic data (i.e., removes all cases where A-A, B-B, C-C, etc.), resulting in a set of samples 436 having probabilities of belonging to a different stratigraphic unit. In step 438, a stratigraphic directionality is determined, i.e., the deep-to-shallow or the shallow-to-deep. This means that the process discards in step 440, for the shallow-to-deep direction, the pairs where the observed depth is larger than the predicted stratigraphic unit base depth, of the predicted stratigraphic unit within the well (e.g., discard A-D, B-F, B-C, etc.), or, for the shallow-to-deep case, discards in step 442 the pairs where the observation (sample) depth is less than the top depth of the predicted stratigraphic unit within the well (e.g., discard F-A, H-E, C-B, etc.). In step 444, the method assesses the well caving or other cross-contamination while in step 446 the method asses the cuttings mixing, local reworking and sand injections based on the constructed pairs 440 and 442, respectively.

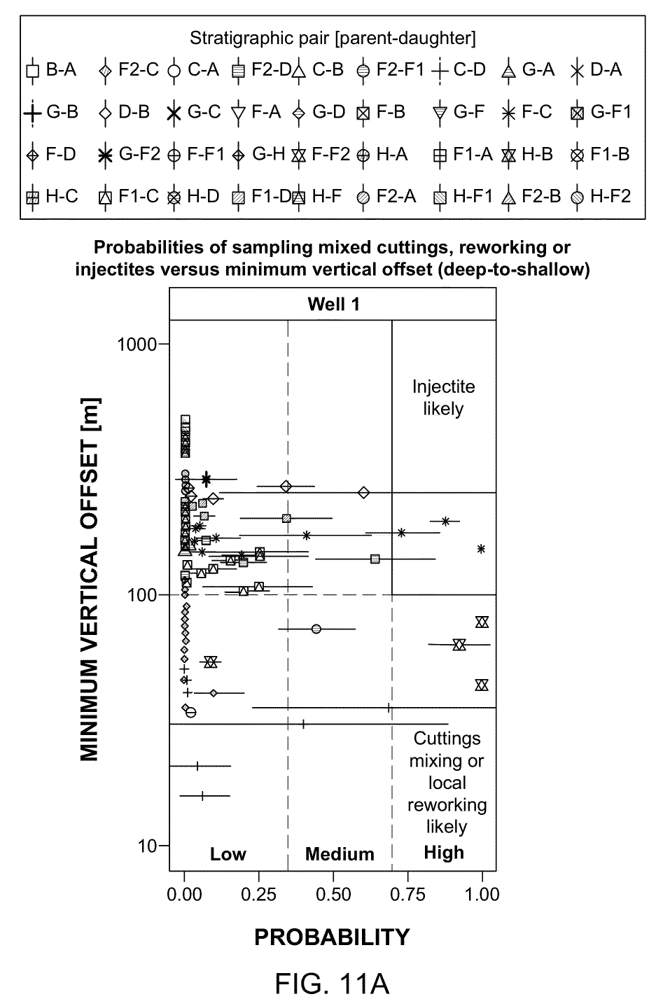

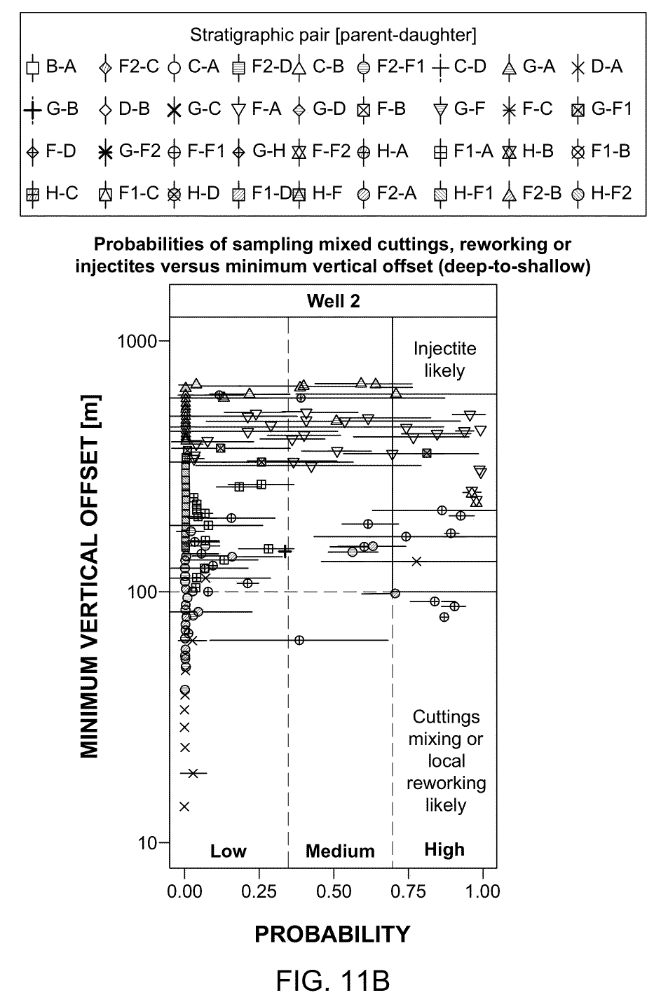

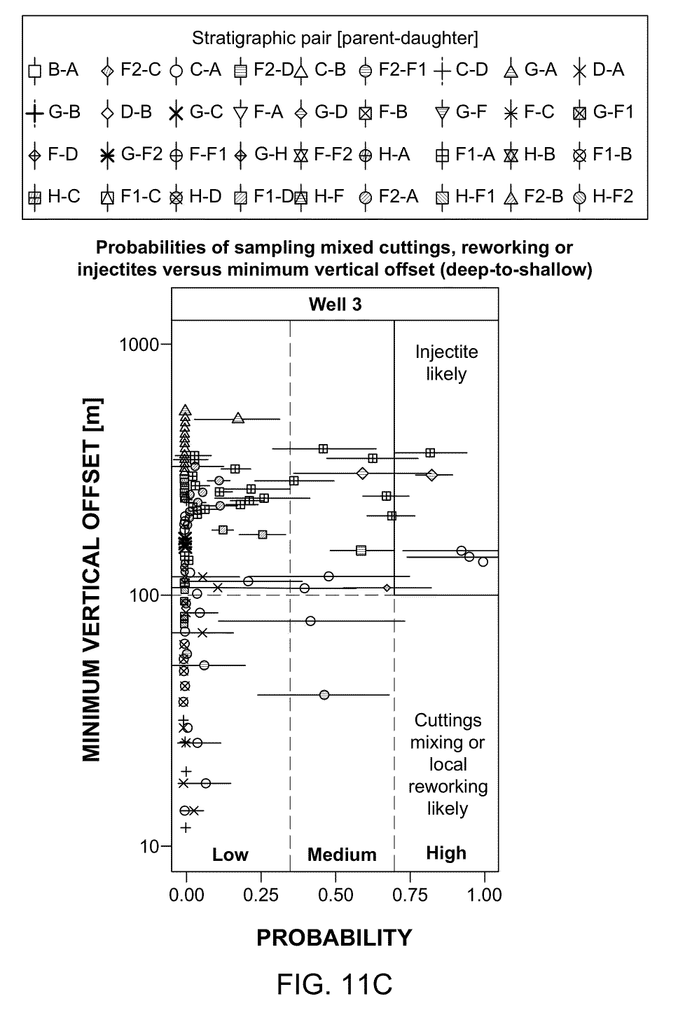

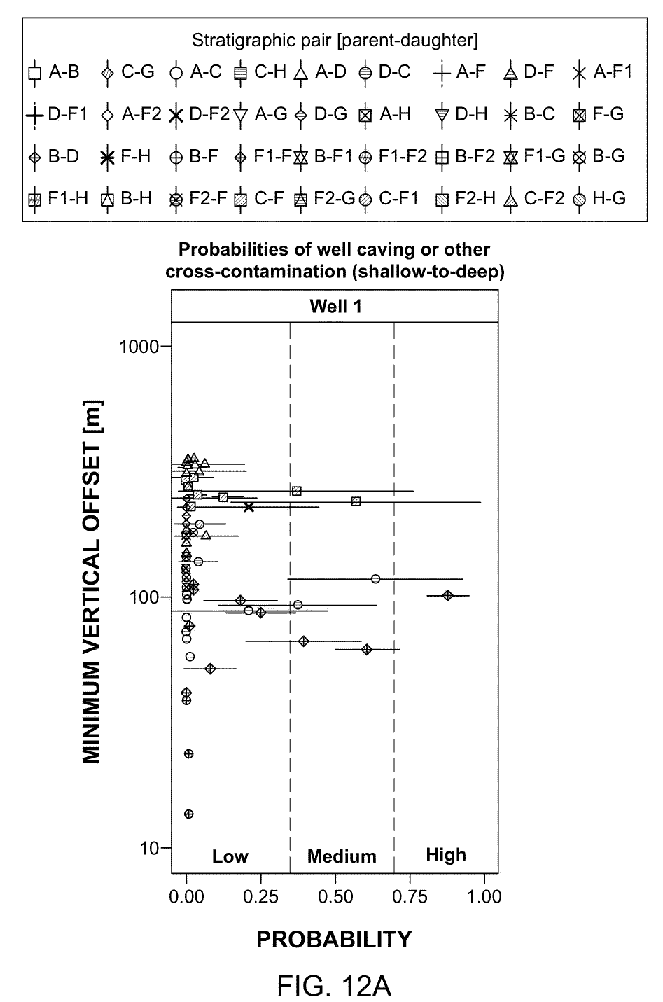

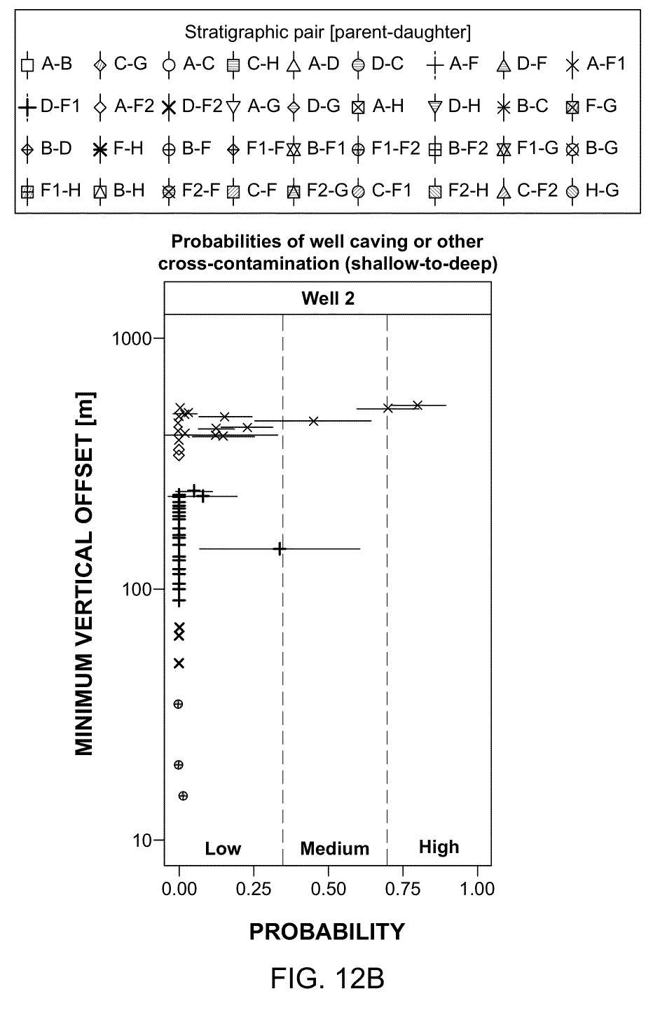

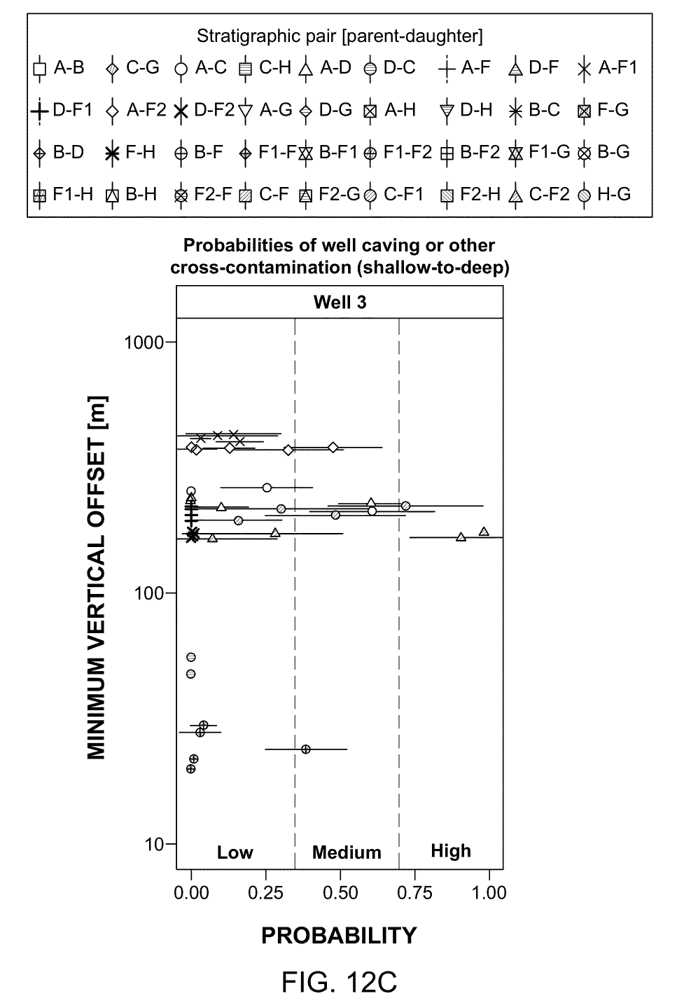

The visualizing step 316 discussed above with regard to FIG. 3 may be implemented as one or more sub-steps. For example, it is possible according to a sub-step to visualize the results as cross-plots of the probability of daughter samples belonging to parent stratigraphic units versus the minimum vertical offset to the top of the parent stratigraphic unit in each well, where the stratigraphic tops may be derived directly from biostratigraphic analysis from samples within the well(s), or from geophysical data interpretation. In this respect, FIGS. 11A to 11C show data points for the deep-to-shallow case and each data point corresponds to a pair of stratigraphic units, predicted-observed. High probability (e.g., a probability higher than 0.7) pairs with large vertical offsets between the sampled depth and the top of the predicted stratigraphic package corresponds to the zone of injection (injectites) whereas high probability pairs with low vertical offsets correspond to cuttings mixing or local reworking. FIGS. 12A to 12C show the data points for the shallow-to-deep case.

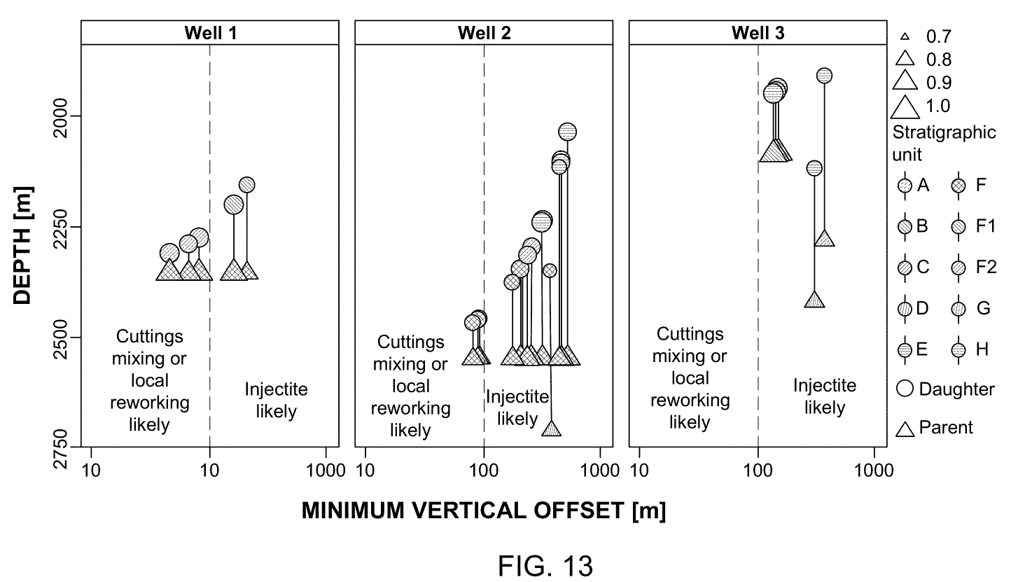

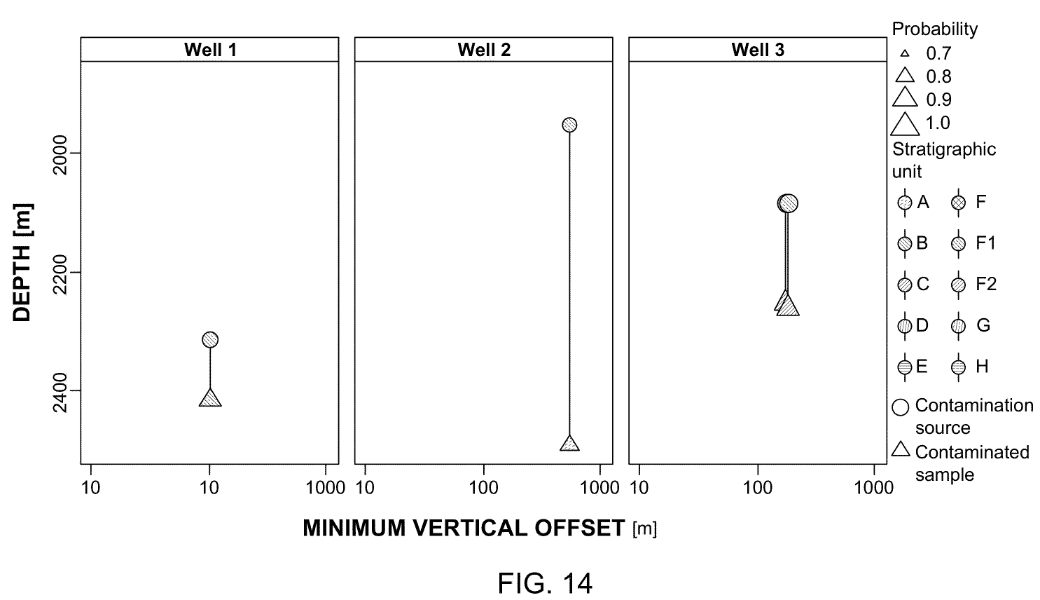

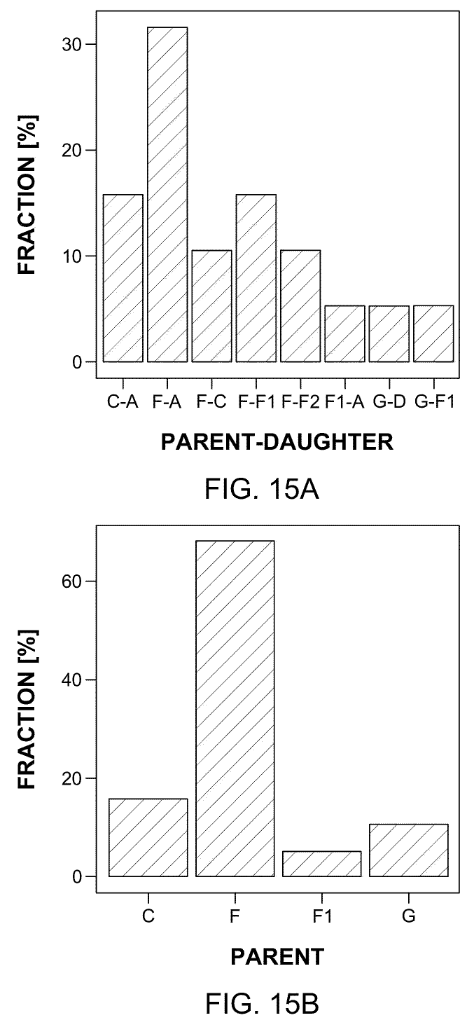

In another or additional sub-step, the method visualizes the results on a well log basis, filtered for the high probability samples belonging to an underlying/overlying stratigraphic unit and including the minimum vertical offset, as illustrated in FIGS. 13 and 14. It is noted that downward cross-contamination is rare for this case (FIG. 14). A third possible sub-step or output for the visualizing step is generating summary statistics for the total number of injectites predicted in each well and across the field, which is illustrated in FIGS. 15A and 15B. For example, these figures show a summary of the fractions of high probability injectite parent-daughter and parent occurrences in the demonstration dataset. Stratigraphic unit F is the primary source of injection. Stratigraphic unit F sand is relatively frequently observed in stratigraphic unit A. It is highly likely that stratigraphic unit F has injected into stratigraphic unit A. The injectite(s) truncate the intervening stratigraphic units if present (e.g., B, C, D and/or E).

The methods discussed above provide a novel and powerful means to quantify the potential for deep-to-shallow cuttings mixing within wells, local depositional reworking, and sand injection. Minimum vertical offset cut off values between injected and reworked sand are set to (but not limited to) 100 m. Low, medium and high probability thresholds are set according to convention (0.35. 0.7). Alternatively, the approach can be applied in terms of shallow-to-deep pairs, which highlight well caving or other causes of cross-contamination. Small offsets between sampled and predicted stratigraphic depth also highlight any discrepancies between stratigraphic interpretations derived from geophysical data versus the ground-truth sample data. Most samples in the demonstration dataset exhibit low probabilities of belonging to a stratigraphic unit other than the a priori assumption. This provides additional, qualitative confidence that the geochemical data are indeed robust fingerprints for each stratigraphic unit.

Thus, the methods discussed above advantageously use elemental geochemical fingerprints to identify high probability deep-to-shallow and shallow-to-deep stratigraphic pairs. The statistical methods of FIGS. 3 and 4 may be fully automated and offer a means to rapidly screen large numbers of analyses for high probability injection. Fractionation of heavy minerals within flows during sand injection is a potential source of uncertainty. However, the proposed method is advantageous because it (1) determines and harnesses the most significant fingerprints for each stratigraphic unit in the system, (2) selects a range of fingerprint elements which partition into different heavy minerals of varying densities and hardness, (3) is based upon comparison of sub-populations which capture natural compositional variation related to hydrodynamic sorting within flows, and (4) defines injection probabilistically.

This approach is applicable (but not limited) to high throughput analysis of several 10 s to 100 s analyses, it offers an order of magnitude improved resolution versus the geophysical data, is deployable on drill cuttings, and does not require (although is applicable to) rock core. The present approach additionally constrains the potential source of injectites and minimum vertical length of injectites. This is desired for estimation of reservoir volume and connectivity. The present approach also identifies potential seal breaches. Whilst the given use-cases (a) to (d) are focused primarily on hydrocarbon exploration and development activities, the workflow is applicable to any drilling activity through layered rocks (e.g., sedimentary strata). The potential to determine cross-contamination and caving is relevant to any drilling activity in the subsurface.

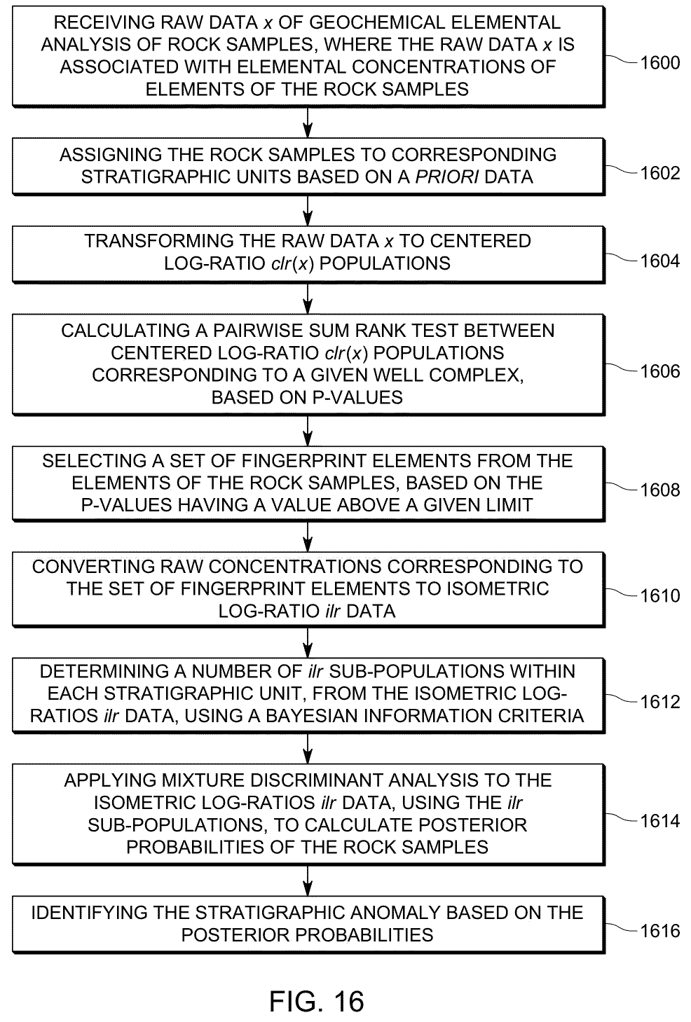

A method that applies multivariate statistical analysis of major and trace element XRF data 404 to fingerprint stratigraphic units in the subsurface, based on the novel concepts discussed above, is now discussed with regard to FIG. 16. This method for determining a stratigraphic anomaly in a subsurface of the earth includes a step 1600 of receiving raw data x 404 of geochemical elemental analysis of rock samples, where the raw data x 404 is associated with elemental concentrations of elements of the rock samples, a step 1602 of assigning the rock samples to corresponding stratigraphic units based on a priori data 402, a step 1604 of transforming the raw data x 404 to a centred log-ratios clr(x) dataset 412, a step 1606 of calculating p-values with a pairwise sum rank test between populations of the centred log-ratios clr(x) dataset 412 corresponding to a given well complex, a step 1608 of selecting a set of fingerprint elements 420 from the elements of the rock samples, based on the p-values having a value above a given limit, a step 1610 of converting raw concentrations corresponding to the set of fingerprint elements 420 to isometric log-ratios ilr data 424, a step 1612 of determining a number of ilr sub-populations 428 within each stratigraphic unit, from the isometric log-ratios ilr data 424, using a Bayesian information criteria, a step 1614 of applying mixture discriminant analysis to the isometric log-ratios ilr data 424, using the ilr sub-populations 428, to calculate posterior probabilities 432 of the rock samples, and a step 1616 of identifying the stratigraphic anomaly based on the posterior probabilities 432.

The stratigraphic anomaly is related to one of sandstone injectite, drill cuttings cross-contamination, well caving, and local reworking. The method may further includes a step of retaining, based on the calculated posterior probabilities of the rock samples, only pairs that define shallow samples, daughters, belonging to underlying stratigraphic units, parents, or only pairs that define deep samples belonging to overlying stratigraphic units. Further, the method may further include a step of plotting the daughters and the parents as cross-plots of a probability that the daughters belong to the parents versus a minimum vertical offset to corresponding tops of the parents. The step of determining may use spherical and diagonal finite Gaussian mixture models. The step of applying may calculate the posterior probabilities of the rock samples as a product of a prior probability and a likelihood, where the likelihood is a probability density function that represents a likelihood that an observed sample belongs to a given stratigraphic unit. The well complex includes plural wells and the rock samples are taken from each well of the plural wells, or includes plural wells and the rock samples are taken from different stratigraphic units of single wells of the plural wells, or includes plural wells and each rock sample is taken from a corresponding strata of the plural wells. The stratigraphic anomaly is identified without seismic data. The method may additionally include a step of assigning each rock sample to a predicted stratigraphic unit based on the calculated posterior probabilities, and a step of determining a stratigraphic directionality by removing all cases where the predicted stratigraphic unit matches the stratigraphic units from the a priori data.

According to another embodiment, there is a method for determining sandstone injectites in a subsurface of the earth, and the method includes a step 1600 of receiving raw data x 404 of geochemical elemental analysis of rock samples, wherein the raw data x 404 is associated with elemental concentrations of elements of the rock samples, a step 1606 of calculating p-values with a Wilcoxon pairwise sum rank test between populations of the raw data x corresponding to a given well complex, a step 1608 of selecting a set of fingerprint elements 420 from the elements of the rock samples, based on the p-values having a value above a given limit, a step 1610 of converting raw concentrations corresponding to the set of fingerprint elements 420 to isometric log-ratios ilr data 424, a step 1614 of applying mixture discrimannt analysis to the isometric log-ratios ilr data 424, using ilr sub-populations (428), to calculate posterior probabilities 432 of the rock samples, and a step 1616 of identifying the sandstone injectite based on the posterior probabilities 432.

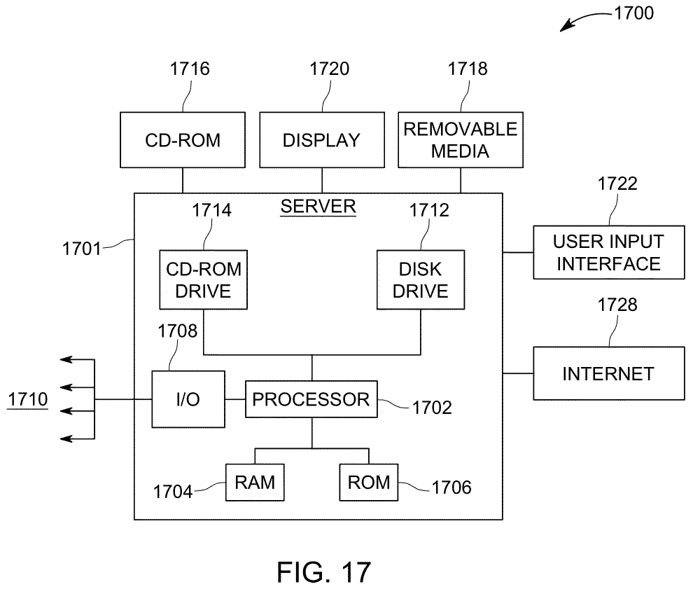

The above methods and others may be implemented in a computing system specifically configured to calculate the subsurface image. An example of a representative computing system capable of carrying out operations in accordance with the exemplary embodiments is illustrated in FIG. 17. Hardware, firmware, software or a combination thereof may be used to perform the various steps and operations described herein.

The computing system 1700 suitable for performing the activities described in the exemplary embodiments may include a server 1701. Such a server 1701 may include a central processor (CPU) 1702 coupled to a random access memory (RAM) 1704 and to a read-only memory (ROM) 1706. ROM 1706 may also be other types of storage media to store programs, such as programmable ROM (PROM), erasable PROM (EPROM), etc. Processor 1702 may communicate with other internal and external components through input/output (I/O) circuitry 1708 and bussing 1710, to provide control signals and the like. Processor 1702 carries out a variety of functions as are known in the art, as dictated by software and/or firmware instructions.

The server 1701 may also include one or more data storage devices, including a disk drive 1712, CD-ROM drives 1714, and other hardware capable of reading and/or storing information such as DVD, etc. In one embodiment, software for carrying out the above-discussed steps may be stored and distributed on a CD-or DVD-ROM 1716, removable memory device 1718 or other form of media capable of portably storing information. These storage media may be inserted into, and read by, devices such as the CD-ROM drive 1714, the disk drive 1712, etc. The server 1701 may be coupled to a display 1720, which may be any type of known display or presentation screen, such as LCD, LED displays, plasma displays, cathode ray tubes (CRT), etc. A user input interface 1722 is provided, including one or more user interface mechanisms such as a mouse, keyboard, microphone, touchpad, touch screen, voice-recognition system, etc.

The server 1701 may be coupled to other computing devices, such as landline and/or wireless terminals, via a network. The server may be part of a larger network configuration as in a global area network (GAN) such as the Internet 1728, which allows ultimate connection to various landline and/or mobile client devices. The computing device may be implemented on a vehicle that moves across land, on a vessel that moves across a body of water, or in a land-based server.

The disclosed embodiments provide a system and a method for calculating stratigraphic anomalies in the subsurface, based on multivariate statistical analysis of rock sample properties, independent of seismic or other geological data. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### References

Hurst et al. 2011. Physical characteristics of sand injectites, Earth Science Reviews, 106, 3-4.

Pernin et al., 2019. Identifying and de-risking near-field opportunities through reliable pre-stack broadband attributes: examples from the Paleocene North Sea (UK-Norway) injectites play. Patruno, S., Archer, S. G., Chiarella, D., Howell, J. A., Jackson, C. A.-L. & Kombrink, H. (Eds) Cross-Border Themes in Petroleum Geology I: The North Sea. Geological Society, London, Special Publications, 494, https://doi.org/10.1144/SP494-2019-11.

Kane, 2010. Development and flow structures of sand injectites: The Hind Sandstone Member injectite complex, Carboniferous, UK. Marine and Petroleum Geology, 27, 6, 1200-1215.

Pernin et al., 2019. Identifying and de-risking near-field opportunities through reliable pre-stack broadband attributes: examples from the Paleocene North Sea (UK-Norway) injectites play. Patruno, S., Archer, S. G., Chiarella, D., Howell, J. A., Jackson, C. A.-L. & Kombrink, H. (Eds) Cross-Border Themes in Petroleum Geology I: The North Sea. Geological Society, London, Special Publications, 494, https://doi.org/10.1144/SP494-2019-11.

Murphy, M., A., Salvador, A., 1999. International Stratigraphic Guide; An abridged version. International Union of Geological Sciences, 22(4): 255-271.

Martin-Fernandez, J.A., Barceló-Vidal, C., Pawlowsky-Glahn, V., 2003. Dealing with Zeros and Missing Values in Compositional Data Sets Using Nonparametric Imputation. Mathematical Geology, 35(3): 253-278.

Ramkumar, M. (2015). Toward standardization of terminologies and recognition of chemostratigraphy as a formal stratigraphic method. In M. Ramkumar (Ed.), Chemostratigraphy — Concepts, techniques, and applications (Chap. 1, pp. 1-22). Amsterdam: Elsevier.

Egozcue, J.J., Pawlowsky-Glahn, V., Mateu-Figueras, G., Barceló-Vidal, C., 2003. Isometric Logratio Transformations for Compositional Data Analysis. Mathematical Geology, 35(3): 279-300.

Fraley, C., Raftery, A.E., 2002. Model-Based Clustering, Discriminant Analysis, and Density Estimation. Journal of the American Statistical Association, 97(458): 611-631.

Scrucca, L., Fop, M., Murphy, T.B., Raftery, A.E., 2016. mclust 5: Clustering, Classification and Density Estimation Using Gaussian Finite Mixture Models. R j, 8(1): 289-317.

Hastie, T., Tibshirani, R., 1996. Discriminant Analysis by Gaussian Mixtures. Journal of the Royal Statistical Society: Series B (Methodological), 58(1): 155-176.

What is claimed is:

1. A method for determining a stratigraphic anomaly in a subsurface of the earth, the method comprising:
receiving raw data x corresponding to a geochemical elemental analysis of rock samples, wherein the raw data x is associated with elemental concentrations of elements of the rock samples;
assigning the rock samples to corresponding stratigraphic units of the subsurface based on a priori data;
transforming the raw data x to a centred log-ratios clr(x) dataset;
calculating p-values with a pairwise sum rank test between populations of the centred log-ratios clr(x) dataset, corresponding to a given well complex;
selecting a set of fingerprint elements from the elements of the rock samples, based on the p-values having a value above a given limit;
converting raw concentrations corresponding to the set of fingerprint elements to isometric log-ratios ilr data;
determining a number of ilr sub-populations within each stratigraphic unit, from the isometric log-ratios ilr data, using a Bayesian information criteria;
applying mixture discriminant analysis to the isometric log-ratios ilr data, using the ilr sub-populations, to calculate posterior probabilities of the rock samples; and
identifying the stratigraphic anomaly based on the posterior probabilities.

2. The method of claim 1, wherein the stratigraphic anomaly is related to one of sandstone injectite, drill cuttings cross-contamination, well caving, and local reworking.

3. The method of claim 1, further comprising:
retaining, based on the calculated posterior probabilities of the rock samples, only pairs that define shallow samples, daughters, belonging to underlying stratigraphic units, parents, or only pairs that define deep samples belonging to overlying stratigraphic units.

4. The method of claim 3, further comprising:
plotting the daughters and the parents as cross-plots of a probability that the daughters belong to the parents versus a minimum vertical offset to corresponding tops of the parents.

5. The method of claim 1, wherein the step of determining uses spherical and diagonal finite Gaussian mixture models.

6. The method of claim 1, wherein the step of applying calculates the posterior probabilities of the rock samples as a product of a prior probability and a likelihood, where the likelihood is a probability density function that represents a chance that an observed sample belongs to a given stratigraphic unit.

7. The method of claim 1, wherein the well complex includes plural wells and the rock samples are taken from each well of the plural wells.

8. The method of claim 1, wherein the well complex includes plural wells and the rock samples are taken from different stratigraphic units of single wells of the plural wells.

9. The method of claim 1, wherein the well complex includes plural wells and each rock sample is taken from a corresponding strata of all of the plural wells.

10. The method of claim 1, wherein the stratigraphic anomaly is identified based on no seismic data.

11. The method of claim 1, further comprising:
assigning each rock sample to a predicted stratigraphic unit based on the calculated posterior probabilities; and
determining a stratigraphic directionality by removing all cases where the predicted stratigraphic unit matches the stratigraphic units from the a priori data.

12. A computing device for determining a stratigraphic anomaly in a subsurface of the earth, the computing device comprising:
an input/output interface configured to receive raw data x of geochemical elemental analysis of rock samples, wherein the raw data x is associated with elemental concentrations of elements of the rock samples; and
a processor connected to the input/output interface and configured to
assign the rock samples to corresponding stratigraphic units based on a priori data,
transform the raw data x to a centred log-ratios clr(x) dataset,
calculate p-values with a pairwise sum rank test between populations of the centred log-ratios clr(x) dataset, corresponding to a given well complex,
select a set of fingerprint elements from the elements of the rock samples, based on the p-values having a value above a given limit,
convert raw concentrations corresponding to the set of fingerprint elements to isometric log-ratios ilr data,
determine a number of ilr sub-populations within each stratigraphic unit, from the isometric log-ratios ilr data, using a Bayesian information criteria,
apply mixture discriminant analysis to the isometric log-ratios ilr data, using the ilr sub-populations, to calculate posterior probabilities of the rock samples, and
identify the stratigraphic anomaly based on the posterior probabilities.

13. The computing device of claim 12, wherein the stratigraphic anomaly is related to one of sandstone injectite, drill cuttings cross-contamination, well caving, and local reworking.

14. The computing device of claim 12, wherein the processor is further configured to:
retain, based on the calculated posterior probabilities of the rock samples, only pairs that define shallow samples, daughters, belonging to underlying stratigraphic units, parents, or only pairs that define deep samples belonging to overlying stratigraphic units.

15. The computing device of claim 14, wherein the processor is further configured to:
plot the daughters and the parents as cross-plots of a probability that the daughters belong to the parents versus a minimum vertical offset to corresponding tops of the parents.

16. The computer device of claim 12, wherein the processor is further configured to calculate the posterior probabilities of the rock samples as a product of a prior probability and a likelihood, where the likelihood is a probability density function that represents a chance that an observed sample belongs to a given stratigraphic unit.

17. The computer device of claim 12, wherein the well complex includes plural wells and the rock samples are taken from each well of the plural wells.

18. The computer device of claim 12, wherein the well complex includes plural wells and the rock samples are taken from different stratigraphic units of single wells of the plural wells.

19. The computer device of claim 12, wherein the well complex includes plural wells and each rock sample is taken from a corresponding strata of all of the plural wells.

20. A method for determining sandstone injectites in a subsurface of the earth, the method comprising:
receiving X-ray fluorescence raw data x of geochemical elemental analysis of rock samples, wherein the raw data x is associated with elemental concentrations of elements of the rock samples;
calculating p-values with a Wilcoxon pairwise sum rank test between populations of the raw data x corresponding to a given well complex;
selecting a set of fingerprint elements from the elements of the rock samples, based on the p-values having a value above a given limit;
converting raw concentrations corresponding to the set of fingerprint elements to isometric log-ratios ilr data;
applying mixture discriminant analysis to the isometric log-ratios ilr data, using ilr sub-populations, to calculate posterior probabilities of the rock samples; and
identifying the sandstone injectite based on the posterior probabilities.
